SEQUENCE LISTING (21 sequences)

SEQ ID NO: 1 - DNA, 20 bp, Artificial Sequence
  Description of Artificial Sequence Synthetic primer
tcgacgtgggttgcraaacg

SEQ ID NO: 2 - DNA, 21 bp, Artificial Sequence
  Description of Artificial Sequence Synthetic primer
tatgaccgttgattcgatacc

SEQ ID NO: 3 - DNA, 31 bp, Artificial Sequence
  Description of Artificial Sequence Synthetic probe
taaatataaatgcaaacgatgaaaactttgc

SEQ ID NO: 4 - DNA, 22 bp, Artificial Sequence
  Description of Artificial Sequence Synthetic primer
tacctacccttgacatacagtg

SEQ ID NO: 5 - DNA, 18 bp, Artificial Sequence
  Description of Artificial Sequence Synthetic primer
cttcctccggtttgtcac

SEQ ID NO: 6 - DNA, 25 bp, Artificial Sequence
  Description of Artificial Sequence Synthetic probe
ccagcatgtgatggtggggactcta

SEQ ID NO: 7 - DNA, 1097 bp, Legionella pneumophila
atgtgggcagaagtattatccagcgatgcctttgcgcgctttgaggaggaaggtattttcaaccccaaaactggacatgactttttaaaatccattctggaggtaggcggctcaagaaaagcagccgatgcttttgttgaattcagaggaagacccgcgacgattgatgccttgctgcgccataacgggattttataaaaacaggacttgcgccccccaatcctctcggtaaaacattttttgccttgaccttggggttttccgtaagtcctgaaaatatattcggtatgctggcgggattttgcttacatgccggcattttatgttataattaaagtgtacagaatggggggcgacctggcttcgacgtgggttgcaaaaccggaagtgcatgccgagaaggagatctctcgtaaataagactcaattaaatataaatgcaaacgatgaaaactttgctggtggggaagctatcgctgcctaataagcactttagttaaaccatcactgtgtactggccaataaacccagtatcccgttcgaccgagcccgcttatcggtatcgaatcaacggtcataagagataagctagcgtcctaatctatcccgggttatggcgcgaaactcagggaatcgctgtgtatcatcctgcccgtcggaggagccacagttaaattcaaaagacaaggctatgcatgtagagctaaaggcagaggacttgcggacgcgggttcgattcccgccgcctccaccaattcattatccgatacagtccaataccgggtctttcccaaatacctgaatcttctacacatcttgtttattccaaacaaacatgatcaaatcacctctttttgaggtatgtatggacttagcagttgaagatactacagcatggtcggaagctatttttggttcagttgctttaggggataaacgacttactcgtcggttaattcaaataggcaaacaattatcatcgacgcctggtggttctctttcaggaagttgtggagggcaggatgcgcttatagaaggtagttatcgttttttacgaaacaaacgagtcacagcgaatcaaattgcagagggtggtt

SEQ ID NO: 8 - DNA, 1475 bp, Legionella pneumophila
aactgaagagtttgatcctggctcagattgaacgctggcggcatgcttaacacatgcaagtcgaacggcagcattgtctagcttgctagacagatggcgagtggcgaacgggtgagtaacgcgtaggaatatgccttgaagagggggacaacttggggaaactcaagctaataccgcataatgtctgaggacgaaagctggggaccttcgggcctggcgctttaagattagcctgcgtccgattagctagttggtggggtaagggcctaccaaggcgacgatcggtagctggtctgagaggatgaccagccacactggaactgagacacggtccagactcctacgggaggcagcagtggggaatattggacaatgggggcaaccctgatccagcaatgccgcgtgtgtgaagaaggcctgagggttgtaaagcactttcagtggggaggagggttgataggttaagagctgattaactggacgttacccacagaagaagcaccggctaactccgtgccagcagccgcggtaatacggagggtgcgagcgttaatcggaattactgggcgtaaagggtgcgtaggtggttgattaagttatctgtgaaattcctgggcttaacctgggacggtcagataatactggttgactcgagtatgggagagggtagtggaatttccggtgtagcggtgaaatgcgtagagatcggaaggaacaccagtggcgaaggcggctacctggcctaatactgacactgaggcacgaaagcgtggggagcaaacaggattagataccctggtagtccacgctgtaaacgatgtcaactagctgttggttatatgaaaataattagtggcgcagcaaacgcgataagttgaccgcctggggagtacggtcgcaagattaaaactcaaaggaattgacgggggcccgcacaagcggtggagcatgtggtttaattcgatgcaacgcgaagaaccttacctacccttgacatacagtgaattttgcagagatgcattagtgccttcgggaacactgatacaggtgctgcatggctgtcgtcagctcgtgtcgtgagatgttgggttaagtcccgtaacgagcgcaacccttatccttagttgccagcatgtgatggtggggactctaaggagactgccggtgacaaaccggaggaaggcggggatgacgtcaagtcatcatggcccttacgggtagggctacacacgtgctacaatggccgatacagagggcggcgaaggggcgacctggagcaaatccttaaaagtcggtcgtagtccggattggagtctgcaactcgactccatgaagtcggaatcgctagtaatcgcgaatcagcatgtcgcggtgaatacgttcccgggccttgtacacaccgcccgtcacaccatgggagtgggttgcaccagaagtagatagtctaaccttcggggggacgtttaccacggtgt

SEQ ID NO: 9 - DNA, 22 bp, Artificial Sequence
  Description of Artificial Sequence Synthetic probe
caaccagccgctgctgacggtc

SEQ ID NO: 10 - DNA, 20 bp, Artificial Sequence
  Description of Artificial Sequence Synthetic primer
tcgacgtgggttgcraaacg

SEQ ID NO: 11 - DNA, 20 bp, Artificial Sequence
  Description of Artificial Sequence Synthetic primer
tcgacgtgggttgcaaaacg

SEQ ID NO: 12 - DNA, 20 bp, Artificial Sequence
  Description of Artificial Sequence Synthetic primer
tcgacgtgggttgctaaacg

SEQ ID NO: 13 - DNA, 20 bp, Artificial Sequence
  Description of Artificial Sequence Synthetic primer
tcgacgtgggttgccaaacg

SEQ ID NO: 14 - DNA, 20 bp, Artificial Sequence
  Description of Artificial Sequence Synthetic primer
tcgacgtgggttgcgaaacg

SEQ ID NO: 15 - DNA, 201 bp, Legionella pneumophila
tacctacccttgacatacagtgaattttgcagagatgcattagtgccttcgggaacactgatacaggtgctgcatggctgtcgtcagctcgtgtcgtgagatgttgggttaagtcccgtaacgagcgcaacccttatccttagttgccagcatgtgatggtggggactctaaggagactgccggtgacaaaccggaggaag

SEQ ID NO: 16 - DNA, 216 bp, Legionella pneumophila
tcgacgtgggttgcaaaaccggaagtgcatgccgagaaggagatctctcgtaaataagactcaattaaatataaatgcaaacgatgaaaactttgctggtggggaagctatcgctgcctaataagcactttagttaaaccatcactgtgtactggccaataaacccagtatcccgttcgaccgagcccgcttatcggtatcgaatcaacggtcata

SEQ ID NO: 17 - DNA, 20 bp, Artificial Sequence
  Description of Artificial Sequence Synthetic primer
gcttcagtaccttcggcttg

SEQ ID NO: 18 - DNA, 20 bp, Artificial Sequence
  Description of Artificial Sequence Synthetic primer
ttgcaggcatctctgacaac

SEQ ID NO: 19 - DNA, 21 bp, Artificial Sequence
  Description of Artificial Sequence Synthetic probe
tggctcttggcggtccagatg

SEQ ID NO: 20 - DNA, 150 bp, Artificial Sequence
  Description of Artificial Sequence Synthetic polynucleotide
gcttcagtaccttcggcttgtggctcttggcggtccagatgctttcttattagaatctatcttcaataacaccatatggggtcatcttgaattgcctgtaagtcgtacaaacgaggaactcatatgccgtgttgtcagagatgcctgcaa

SEQ ID NO: 21 - DNA, 577 bp, Artificial Sequence
  Description of Artificial Sequence Synthetic polynucleotide
taaccccgcgataaagacagaagattatgcatacgagatcaaaggagccggccttttctctagagatctcttattttccttgaagtcacctgtttatgttaaagcaggtgagcaggtatacattcagtacgatctgaacaaaagcaatgcagaacttgctctcgactatggttttgtggaatcaaaccctaaacggaactcatatactttaacaatagagataccagaatcagacccattctttggggataagttggatattgctgagagtaacaagatgggtgagaccggatactttgacatagtagacggccagactcttcccgctggtatgcttcagtaccttcggcttgtggctcttggcggtccagatgctttcttattagaatctatcttcaataacaccatatggggtcatcttgaattgcctgtaagtcgtacaaacgaggaactcatatgccgtgttgtcagagatgcctgcaaatctgctctgtctggttttgatacgaccattgaagaggatgagaagcttctggacaaaggaaagcttgagcctaggttggaaatggctctcaag

(19) United States
(12) Patent Application Publication
(10) Pub. No.: US 2022/0145367 A1
(43) Pub. Date: May 12, 2022

(54) METHODS FOR DETECTING LEGIONELLA

(71) Applicant: Quest Diagnostics Investments LLC, Secaucus, NJ (US)

(72) Inventor: Erik P. Johnson, Carlsbad, CA (US)

(73) Assignee: Quest Diagnostics Investments LLC, Secaucus, NJ (US)

(21) Appl. No.: 17/427,014

(22) PCT Filed: Jan. 30, 2020
(86) PCT No.: PCT/US2020/015950

§ 371 Date: Jul. 29, 2021

Related U.S. Application Data
(60) Provisional application No. 62/799,424, filed on Jan. 31, 2019.

(51) Int. Cl. C12Q 1/689; C12Q 1/6818; A61K 31/505; A61K 31/496; A61K 31/4375; A61K 31/5383; A61K 31/4709; A61K 31/407; A61K 31/4162; A61K 31/427; A61K 39/02; A61K 31/7052; A61K 31/7048; A61P 31/04

(52) U.S. Cl. CPC C12Q 1/689; C12Q 2600/166; A61K 31/505; A61K 31/496; A61K 31/4375; A61K 31/5383; A61K 31/4709; A61K 31/407; A61K 31/4162; A61K 31/427; A61K 39/0208; A61K 31/7052; A61K 31/7048; A61P 31/04; C12Q 1/6818

(57) ABSTRACT

The present disclosure provides methods for determining whether a patient exhibiting pneumonia-like symptoms will benefit from treatment with therapeutic agents that inhibit Legionella sp. These methods are based on detecting Legionella sp. and/or Legionella pneumophila in a biological sample. Kits for use in practicing the methods are also provided.

### TECHNICAL FIELD

The present disclosure provides methods for determining whether a patient exhibiting pneumonia-like symptoms will benefit from treatment with therapeutic agents that inhibit Legionella sp. and/or Legionella pneumophila. These methods are based on detecting Legionella sp. and Legionella pneumophila in a biological sample by assaying for the presence of the ssrA gene and the 16S gene, respectively. Kits for use in practicing the methods are also provided.

### BACKGROUND

The following description of the background of the present disclosure is provided simply to aid the reader in understanding the disclosure and is not admitted to describe or constitute prior art to the present disclosure.

Legionellae are facultative intracellular Gram-negative bacteria found in soil and water environments, where they can parasitize and proliferate within protozoa. As a result, they are common contaminates of artificial water systems, including air-conditioning systems, cooling towers, and hot tubs. Legionellae are also capable of replication in mammalian alveolar macrophages and epithelial cells. Once aerosolized, the bacteria can enter the human respiratory tract and cause community-acquired, travel-acquired, and nosocomially-acquired pneumonia. In some cases, Legionellae can cause Legionnaires' disease, a severe form of pneumonia, or Pontiac fever, a milder, flu-like illness.

There are currently 50 known species comprising about 70 distinct serogroups in the genus Legionella. Legionella pneumophila serogroup 1 accounts for the majority of infections in humans, but association with human disease has been reported for greater than 20 of the species within the genus. Identification of infections caused by L. pneumophila is particularly important because this species of Legionella is associated with disease resulting in severe morbidity and mortality.

Culture is considered the “gold standard” for detection of Legionallae. However, legionellae are slow-growing and fastidious organisms. Serological diagnosis is also commonly used; however serological diagnoses can only be made retrospectively and rarely influence treatment of the patient. Urine antigen tests are rapid tests for the diagnosis of Legionnaires' disease, but are limited by their ability to detect only a limited number of Legionella pneumophila serogroups. Existing nucleic acid amplification tests are susceptible to detection of non-Legionella bacterium including Pseudomonas species and Enterobacter species.

Thus, there is a substantial need for more robust, sensitive, and specific methods that can rapidly detect and discriminate between Legionella sp. and Legionella pneumophila in a single biological sample.

### SUMMARY

The present disclosure provides compositions and methods for detecting and discriminating between Legionella sp. and Legionella pneumophila in a single biological sample. In another aspect, the methods and compositions of the present technology are useful in selecting an optimal therapeutic regimen for a subject exhibiting pertussis-like symptoms. Nucleic acid amplification tests (including assays using real-time PCR) are attractive tools for the detection of legionellae in clinical specimens since they are able to detect all legionellae and provide rapid results. These tests are also able to differentiate between L. pneumophila and non-L. pneumophila species. This is important given that more severe morbidity and mortality has been observed with L. pneumophila.

Accordingly, in some aspects, provided herein are methods for detecting the presence of at least one Legionella species in a biological sample, the methods comprising, consisting of, or consisting essentially of: (a) providing a first primer pair suitable for amplifying an ssrA target nucleic acid; providing a second primer pair suitable for amplifying a 16S rRNA target nucleic acid; amplifying the ssrA target nucleic acid and the 16S rRNA target nucleic acid, if present; and detecting one or more amplification products produced in step (c); wherein the presence of the ssrA target nucleic acid identifies the presence of at least one Legionella species, and the presence of the 16S rRNA target nucleic acid identifies the presence of Legionella pneumophila.

In some embodiments of the methods provided herein, the first primer pair comprises at least one degenerate primer. In some embodiments, the first primer pair comprises a first forward primer comprising 5′ TCGACGTGGGTTGCRAAACG 3′ (SEQ ID NO: 1) or a complement thereof. The method of any one of the previous claims, wherein the first primer pair comprises a first reverse primer comprising 5′ TATGACCGTTGATTCGATACC 3′(SEQ ID NO: 2) or a complement thereof. In some embodiments, the second primer pair comprises at least one degenerate primer. In some embodiments, the second primer pair comprises a second forward primer comprising 5′ TACCTACCCTTGACATACAGTG 3′ (SEQ ID NO: 4) or a complement thereof. In some embodiments, second primer pair comprises a second reverse primer comprising 5′ CTTCCTCCGGTTTGTCAC 3′ (SEQ ID NO: 5) or a complement thereof.

In some embodiments, the methods further comprise contacting the biological sample with one or more oligonucleotide probes capable of specifically hybridizing to an amplification product or a complement thereof. In some embodiments, the oligonucleotide probe is detectably labeled. In some embodiments, the detectable label is a fluorescent label. In some embodiments, the fluorescent label is selected from the group consisting of fluorescein, Cy3, Cy5, Cy5.5 tetrachloro-6-car-boxyfluorescein, 2,7-dimethoxy-4,5-dichloro-6-carboxy-fluorescein, Yakima Yellow, Texas Red, TYE 563, ROX, TEX 615, TYE 665, TYE 705, and hexacholoro-6-carboxyfluorescein. In some embodiments, the oligonucleotide probe further comprises at least one quencher. In some embodiments, the quencher is selected from the group consisting of TAMRA, Black Hole Quencher, Deep Dark Quencher, ZEN, Iowa Black FQ, Iowa Black RQ, and DABCYL. In some embodiments, the oligonucleotide probe specifically hybridizes to an ssrA amplification product and wherein the oligonucleotide probe comprises 5′ TAAATATAAATGCAAACGATGAAAACTTTGC 3′(SEQ ID NO: 3) or a complement thereof. In some embodiments, the oligonucleotide probe specifically hybridizes to a 16S rRNA amplification product and wherein the oligonucleotide probe comprises 5′ CCAGCATGTGATGGTGGGGACTCTA 3′(SEQ ID NO: 6) or a complement thereof.

In some embodiments, the methods further comprise admixing exogenous control DNA with the biological sample. In some embodiments, the methods further comprise contacting the biological sample with a third primer pair suitable for amplification of an exogenous control target nucleic acid and amplifying the exogenous control target nucleic acid. In some embodiments, the exogenous control target nucleic acid comprises SEQ ID NO: 20. In some embodiments, the third primer pair consists of a third forward primer comprising 5′ GCTTCAGTACCTTCGGCTTG 3′ (SEQ ID NO: 17) and a third reverse primer comprising 5′ TTGCAGGCATCTCTGACAAC 3′ (SEQ ID NO: 18). In some embodiments, the methods further comprise contacting the biological sample with a third oligonucleotide probe, wherein the third oligonucleotide probe is detectably labeled and comprises 5′ TGGCTCTTGGCGGTCCAGATG 3′ (SEQ ID NO: 19).

In some embodiments of the methods provided herein, the real-time PCR amplification is performed in a direct amplification disc in concert with an integrated thermal cycler.

In some embodiments of the methods provided herein, the biological sample is a bronchoalveolar lavage sample, a bronchial wash sample, a sputum sample, a nasopharyngeal (NP) aspirate or wash sample, a nasal swab, or a bacterial isolate.

In another aspect, provided herein are kits for detecting the presence of at least one Legionella species in a biological sample, the kits comprising, consisting of, or consisting essentially of: (a) a first primer pair that amplifies an ssrA target nucleic acid; (b) a second primer pair that amplifies a 16S rRNA target nucleic acid; (c) a first oligonucleotide probe capable of specifically hybridizing to a segment of the ssrA target nucleic acid; and (d) a second oligonucleotide probe capable of specifically hybridizing to a segment of the 16S rRNA target nucleic acid; wherein the first oligonucleotide probe and the second oligonucleotide probe are detectably labeled.

In some embodiments of the kits provided herein, the kits further comprise a third primer pair that that amplifies a control target nucleic acid. In some embodiments, the first primer pair is capable of specifically hybridizing to a ssrA target nucleic acid comprising nucleotides that are at least 85-95% identical to SEQ ID NO: 7, or a complement thereof. In some embodiments, the second primer pair is capable of specifically hybridizing to a 16S rRNA target nucleic acid comprising nucleotides that are at least 85-95% identical to SEQ ID NO: 8, or a complement thereof.

In some embodiments of the kits provided herein, the first primer pair comprises at least one degenerate primer. In some embodiments, the first primer pair comprises a first forward primer comprising 5′ TCGACGTGGGTTGCRAAACG 3′ (SEQ ID NO: 1) or a complement thereof. In some embodiments, the first primer pair comprises a first reverse primer comprising 5′ TATGACCGTTGATTCGATACC 3′(SEQ ID NO: 2) or a complement thereof. In some embodiments, the second primer pair comprises at least one degenerate primer. In some embodiments, the second primer pair comprises a second forward primer comprising 5′ TACCTACCCTTGACATACAGTG 3′ (SEQ ID NO: 4) or a complement thereof. In some embodiments, the second primer pair comprises a second reverse primer comprising 5′ CTTCCTCCGGTTTGTCAC 3′ (SEQ ID NO: 5) or a complement thereof. In some embodiments, the first nucleic acid probe comprises 5′ TAAATATAAATGCAAACGATGAAAACTTTGC 3′(SEQ ID NO: 3), or a complement thereof. In some embodiments, the second nucleic acid probe comprises 5′ CAACCAGCCGCTGCTGACGGTC 3′ (SEQ ID NO: 9), or a complement thereof.

In some embodiments of the kits provided herein, the detectable label is a fluorescent label. In some embodiments, the fluorescent label is selected from the group consisting of fluorescein, Cy3, Cy5, Cy5.5 tetrachloro-6-car-boxyfluorescein, 2,7-dimethoxy-4,5-dichloro-6-carboxy-fluorescein, Yakima Yellow, Texas Red, TYE 563, ROX, TEX 615, TYE 665, TYE 705, and hexacholoro-6-carboxyfluorescein. In some embodiments, at least one oligonucleotide probe further comprises at least one quencher. In some embodiments, the oligonucleotide probe comprises two quenchers. In some embodiments, the quencher is selected from the group consisting of TAMRA, Black Hole Quencher, Deep Dark Quencher, ZEN, Iowa Black FQ, Iowa Black RQ, and DABCYL.

In one aspect, provided herein is a composition comprising a detectably labeled oligonucleotide probe comprising 5′ TAAATATAAATGCAAACGATGAAAACTTTGC 3′ (SEQ ID NO: 3). In some embodiments, the detectable label is a fluorescent label. In some embodiments, the fluorescent label is selected from the group consisting of fluorescein, Cy3, Cy5, Cy5.5 tetrachloro-6-car-boxyfluorescein, 2,7-dimethoxy-4,5-dichloro-6-carboxy-fluorescein, Yakima Yellow, Texas Red, TYE 563, ROX, TEX 615, TYE 665, TYE 705, and hexacholoro-6-carboxyfluorescein. In some embodiments, the oligonucleotide probe further comprises at least one quencher. In some embodiments, the quencher is selected from the group consisting of TAMRA, Black Hole Quencher, Deep Dark Quencher, ZEN, Iowa Black FQ, Iowa Black RQ, and DABCYL.

Also provided herein are methods for selecting a subject exhibiting pneumonia-like symptoms for treatment with a therapeutic agent that inhibits Legionella pneumophila, the methods comprising, consisting of, or consisting essentially of: (a) contacting a sample isolated from the subject with a first primer pair suitable for amplifying an ssrA target nucleic acid; (b) contacting the sample with a second primer pair suitable for amplifying a 16S rRNA target nucleic acid; (c) amplifying the ssrA target nucleic acid and the 16S rRNA target nucleic acid, if present; and (d) detecting one or more amplification products produced in step (c); and (e) selecting the subject for treatment with a therapeutic agent that inhibits Legionella pneumophila if an amplification product for the 16S rRNA target nucleic acid is detected.

In some aspects, provided herein are methods of treating a subject with a Legionella pneumophila infection, the method comprising, consisting of, or consisting essentially of administering a therapeutic agent that inhibits Legionella pneumophila to a subject selected a method comprising, consisting of, or consisting essentially of: (a) contacting a sample isolated from the subject with a first primer pair suitable for amplifying an ssrA target nucleic acid; (b) contacting the sample with a second primer pair suitable for amplifying a 16S rRNA target nucleic acid; (c) amplifying the ssrA target nucleic acid and the 16S rRNA target nucleic acid, if present; and (d) detecting one or more amplification products produced in step (c); and (e) selecting the subject for treatment with a therapeutic agent that inhibits Legionella pneumophila if an amplification product for the 16S rRNA target nucleic acid is detected.

In some embodiments of the methods provided herein, the first primer pair comprises at least one degenerate primer. In some embodiments, the first primer pair comprises a first forward primer comprising 5′ TCGACGTGGGTTGCRAAACG 3′ (SEQ ID NO: 1) or a complement thereof. The method of any one of the previous claims, wherein the first primer pair comprises a first reverse primer comprising 5′ TATGACCGTTGATTCGATACC 3′(SEQ ID NO: 2) or a complement thereof. In some embodiments, the second primer pair comprises at least one degenerate primer. In some embodiments, the second primer pair comprises a second forward primer comprising 5′ TACCTACCCTTGACATACAGTG 3′ (SEQ ID NO: 4) or a complement thereof. In some embodiments, second primer pair comprises a second reverse primer comprising 5′ CTTCCTCCGGTTTGTCAC 3′ (SEQ ID NO: 5) or a complement thereof.

In some embodiments, the methods further comprise contacting the biological sample with one or more oligonucleotide probes capable of specifically hybridizing to an amplification product or a complement thereof. In some embodiments, the oligonucleotide probe is detectably labeled. In some embodiments, the detectable label is a fluorescent label. In some embodiments, the fluorescent label is selected from the group consisting of fluorescein, Cy3, Cy5, Cy5.5 tetrachloro-6-car-boxyfluorescein, 2,7-dimethoxy-4,5-dichloro-6-carboxy-fluorescein, Yakima Yellow, Texas Red, TYE 563, ROX, TEX 615, TYE 665, TYE 705, and hexacholoro-6-carboxyfluorescein. In some embodiments, the oligonucleotide probe further comprises at least one quencher. In some embodiments, the quencher is selected from the group consisting of TAMRA, Black Hole Quencher, Deep Dark Quencher, ZEN, Iowa Black FQ, Iowa Black RQ, and DABCYL. In some embodiments, the oligonucleotide probe specifically hybridizes to an ssrA amplification product and wherein the oligonucleotide probe comprises 5′ TAAATATAAATGCAAACGATGAAAACTTTGC 3′(SEQ ID NO: 3) or a complement thereof. In some embodiments, the oligonucleotide probe specifically hybridizes to a 16S rRNA amplification product and wherein the oligonucleotide probe comprises 5′ CCAGCATGTGATGGTGGGGACTCTA 3′(SEQ ID NO: 6) or a complement thereof.

In some embodiments, the methods further comprise admixing exogenous control DNA with the biological sample. In some embodiments, the methods further comprise contacting the biological sample with a third primer pair suitable for amplification of an exogenous control target nucleic acid and amplifying the exogenous control target nucleic acid. In some embodiments, the exogenous control target nucleic acid comprises SEQ ID NO: 20. In some embodiments, the third primer pair consists of a third forward primer comprising 5′ GCTTCAGTACCTTCGGCTTG 3′ (SEQ ID NO: 17) and a third reverse primer comprising 5′ TTGCAGGCATCTCTGACAAC 3′ (SEQ ID NO: 18). In some embodiments, the methods further comprise contacting the biological sample with a third oligonucleotide probe, wherein the third oligonucleotide probe is detectably labeled and comprises 5′ TGGCTCTTGGCGGTCCAGATG 3′ (SEQ ID NO: 19).

In some embodiments of the methods provided herein, the real-time PCR amplification is performed in a direct amplification disc in concert with an integrated thermal cycler.

In some embodiments of the methods provided herein, the biological sample is a bronchoalveolar lavage sample, a bronchial wash sample, a sputum sample, a nasopharyngeal (NP) aspirate or wash sample, a nasal swab, or a bacterial isolate.

In some embodiments of the methods provided herein, the therapeutic agent that inhibits Legionella pneumophila is one or more agents selected from the group consisting of fluoroquinolones, carbapenems, macrolide-antibiotics, trimethoprim-sulfamethoxazole, Legionella pneumophila-specific antibodies, and Legionella pneumophila-specific vaccines. In some embodiments of the methods provided herein, the fluoroquinolones are selected from the group consisting of ciprofloxacin, gemifloxacin, levofloxacin, norfloxacin, ofloxacin, rovafloxacin, gatifloxacin, grepafloxacin, temafloxacin, lomefloxacin, sparfloxacin, enoxacin, and moxifloxacin. In some embodiments of the methods provided herein, the carbapenems are selected from the group consisting of imipenem, meropenem, ertapenem, doripenem, panipenem, biapenem, razupenem (PZ-601), tebipenem, lenapenem, tomopenem, and thienpenem (Thienamycin). In some embodiments of the methods provided herein, the Legionella pneumophila-specific vaccine is selected from the group consisting of whole-cell (wP) Legionella pneumophila vaccine and acellular Legionella pneumophila vaccine In some embodiments of the methods provided herein, the macrolide-antibiotics are selected from the group consisting of azithromycin (Zithromax), clarithromycin (Biaxin), erythromycin (E-Mycin, Eryc, Ery-Tab, PCE, Pediazole, Ilosone), and roxithromycin.

### DETAILED DESCRIPTION

The present disclosure provides methods for determining whether a patient exhibiting pneumonia-like symptoms will benefit from treatment with therapeutic agents that inhibit Legionella sp. and/or L. pneumophila. These methods are based on detecting Legionella sp. and/or L. pneumophila in a biological sample by assaying for the presence of the ssrA and 16S rRNA target nucleic acids respectively using real-time PCR. In some embodiments, the methods comprise: (a) providing a first primer pair suitable for amplifying an ssrA target nucleic acid; providing a second primer pair suitable for amplifying a 16S rRNA target nucleic acid; amplifying the ssrA target nucleic acid and the 16S rRNA target nucleic acid, if present; and detecting one or more amplification products produced in step (c); wherein the presence of the ssrA target nucleic acid identifies the presence of at least one Legionella species, and the presence of the 16S rRNA target nucleic acid identifies the presence of Legionella pneumophila. Kits for use in practicing the methods are also provided.

### Definitions

Unless otherwise defined, all technical and scientific terms used herein have the same meaning as commonly understood by one of ordinary skill in the art to which the present technology belongs.

As used herein, unless otherwise stated, the singular forms “a,” “an,” and “the” include plural reference. Thus, for example, a reference to “an oligonucleotide” includes a plurality of oligonucleotide molecules, and a reference to “a nucleic acid” is a reference to one or more nucleic acids.

As used herein, the term “about” in reference to a number is generally taken to include numbers that fall within a range of 1%-10% in either direction (greater than or less than) of the number unless otherwise stated or otherwise evident from the context.

As used herein, the terms “amplify” or “amplification” with respect to nucleic acid sequences, refer to methods that increase the representation of a population of nucleic acid sequences in a sample. Copies of a particular target nucleic acid sequence generated in vitro in an amplification reaction are called “amplicons” or “amplification products”. Amplification may be exponential or linear. A target nucleic acid may be DNA (such as, for example, genomic DNA and cDNA) or RNA. While the exemplary methods described hereinafter relate to amplification using polymerase chain reaction (PCR), numerous other methods such as isothermal methods, rolling circle methods, etc., are well known to the skilled artisan. The skilled artisan will understand that these other methods may be used either in place of, or together with, PCR methods. See, e.g., Saiki, “Amplification of Genomic DNA” in PCR PROTOCOLS, Innis et al., Eds., Academic Press, San Diego, Calif. 1990, pp 13-20; Wharam, et al., Nucleic Acids Res. 29(11):E54-E54 (2001).

An “amplification mixture” as used herein is a mixture of reagents that are used in a nucleic acid amplification reaction, but does not contain primers or sample. An amplification mixture comprises a buffer, dNTPs, and a DNA polymerase. An amplification mixture may further comprise at least one of MgCl₂, KCl, nonionic and ionic detergents (including cationic detergents).

An “amplification master mix” comprises an amplification mixture and primers for amplifying one or more target nucleic acids, but does not contain the sample to be amplified.

The terms “complement”, “complementary” or “complementarity” as used herein with reference to polynucleotides (i.e., a sequence of nucleotides such as an oligonucleotide or a target nucleic acid) refer to the Watson/Crick base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5′.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

The term “substantially complementary” as used herein means that two sequences hybridize under stringent hybridization conditions. The skilled artisan will understand that substantially complementary sequences need not hybridize along their entire length. In particular, substantially complementary sequences may comprise a contiguous sequence of bases that do not hybridize to a target sequence, positioned 3′ or 5′ to a contiguous sequence of bases that hybridize under stringent hybridization conditions to a target sequence.

As used herein, a “cycle threshold” (Ct) for an analyte is the PCR cycle at which the fluorescence signal crosses a specified fluorescence threshold. The Ct depends on the amplification reaction efficiency which includes starting template copy number, organism lysis, PCR amplification, hybridization or cleavage of a fluorogenic probe and sensitivity of detection. The Ct provides a relative measure of the concentration of the target nucleic acid in the PCR reaction. Many factors other than the concentration of the target nucleic acid can impact the absolute value of Ct. However, artifacts from the reaction mix or instrument that change the fluorescence measurements associated with the Ct calculation will result in template-independent changes to the Ct value.

As used herein, the term “detecting” refers to determining the presence of a target nucleic acid in the sample. Detection does not require the method to provide 100% sensitivity and/or 100% specificity.

As used herein, the term “direct amplification” refers to a nucleic acid amplification reaction in which the target nucleic acid is amplified from the sample without prior purification, extraction, or concentration.

As used herein, the term “extraction” refers to any action taken to remove nucleic acids from other (non-nucleic acid) material present in the sample. The term extraction includes mechanical or chemical lysis, addition of detergent or protease, or precipitation and removal of non-nucleic acids such as proteins.

The term “fluorophore” as used herein refers to a molecule that absorbs light at a particular wavelength (excitation frequency) and subsequently emits light of a longer wavelength (emission frequency). The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tm) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the terms “individual”, “patient”, or “subject” can be an individual organism, a vertebrate, a mammal, or a human. In a preferred embodiment, the individual, patient or subject is a human.

As used herein, the term “multiplex PCR” refers to the simultaneous generation of two or more PCR products or amplicons within the same reaction vessel. Each PCR product is primed using a distinct primer pair. A multiplex reaction may further include specific probes for each product that are labeled with different detectable moieties.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides that function as primers or probes are generally at least about 10-15 nucleotides in length or up to about 70, 100, 110, 150 or 200 nucleotides in length, and more preferably at least about 15 to 25 nucleotides in length. Oligonucleotides used as primers or probes for specifically amplifying or specifically detecting a particular target nucleic acid generally are capable of specifically hybridizing to the target nucleic acid.

As used herein, a “Legionella species” or “Legionella sp.” refers to any microbial organism of the Legionella genus. In some embodiments, a Legionella sp. is pathogenic and capable of causing pneumonia, Legionnaire's disease, Pontiac fever, or a related condition in a subject (e.g., a human). Specific pathogens include, for example, L. anisa, L. birminghamensis, L. bozemanii (serogroup 1), L. bozemanii (serogroup 2), L. cardiaca, L. cherrii, L. cincinnatiensis, L. clemsonensis, L. dumoffii, L. feeleii (serogroup 1), L. feeleii (serogroup 2), L. gormanii, L. hackeliae (serogroup 1), L. hackeliae (serogroup 2), L. jordanis, L. lansingensis, L. longbeachae (serogroup 1), L. longbeachae (serogroup 2), L. maceachernii, L. micdadei, L. oakridgensis, L. parisiensis, L. pneumophila (Philadelphia 1), L. pneumophila (Knoxville 1), L. pneumophila (Benidorm 030 E), L. pneumophila (France 5811), L. pneumophila (Allentown 1), L. pneumophila (OLDA), Legionella pneumophila (Oxford 4032 E), Legionella pneumophila (Bellingham), Legionella pneumophila (Heysham 1), Legionella pneumophila (Camperdown 1), Legionella pneumophila (Togus 1), Legionella pneumophila (Bloomington 2), Legionella pneumophila (Los Angeles), L. pneumophila (Portland 1), L. pneumophila (Dallas JE), L. pneumophila (Cambridge 1), L. pneumophila (Chicago 1), L. pneumophila (Chicago 8), L. pneumophila (Concord 3), Legionella pneumophila (IN-23-GI-C2), Legionella pneumophila (Leiden 1), L. pneumophila (797-PA-H), Legionella pneumophila (570-CO-H), Legionella pneumophila (82A3105), Legionella pneumophila (1169-MN-H), Legionella pneumophila (Lansing 3), L. rubrilucens, L. sainthelensi (serogroup 1), L. sainthelensi (serogroup 2), L. tucsonensis, and L. wadsworthii.

A “positive control nucleic acid” or “internal positive amplification control” as used herein is a nucleic acid known to be present in a sample at a certain amount or level. In some embodiments, a positive control nucleic acid is not naturally present in a sample and is added to the sample prior to subjecting the reaction-sample mixture to real-time polymerase chain reaction in the disclosed methods for detecting the presence of pathogenic Legionella sp. in a sample.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of double-stranded DNA (dsDNA). A “reverse primer” anneals to the sense-strand of dsDNA.

Primers are typically at least 10, 15, 18, or 30 nucleotides in length or up to about 100, 110, 125, or 200 nucleotides in length. In some embodiments, primers are preferably between about 15 to about 60 nucleotides in length, and most preferably between about 25 to about 40 nucleotides in length. In some embodiments, primers are 15 to 35 nucleotides in length. There is no standard length for optimal hybridization or polymerase chain reaction amplification. An optimal length for a particular primer application may be readily determined in the manner described in H. Erlich, PCR Technology, PRINCIPLES AND APPLICATION FOR DNA AMPLIFICATION, (1989).

A “primer extension reaction” refers to a synthetic reaction in which an oligonucleotide primer hybridizes to a target nucleic acid and a complementary copy of the target nucleic acid is produced by the polymerase-dependent 3′-addition of individual complementary nucleotides. In some embodiments, the primer extension reaction is PCR.

As used herein, the term “primer pair” refers to a forward and reverse primer pair (i.e., a left and right primer pair) that can be used together to amplify a given region of a nucleic acid of interest.

“Probe” as used herein refers to nucleic acid that interacts with a target nucleic acid via hybridization. A probe may be fully complementary to a target nucleic acid sequence or partially complementary. The level of complementarity will depend on many factors based, in general, on the function of the probe. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. A probe may be used to detect the presence or absence of a methylated target nucleic acid. Probes are typically at least about 10, 15, 20, 25, 30, 35, 40, 50, 60, 75, 100 nucleotides or more in length.

A “probe element” as used herein refers to a stretch of nucleotides that (a) is associated with a primer in that it is connected to or located adjacent to the primer nucleic acid sequence, and (b) specifically hybridizes under stringent conditions to a target nucleic acid sequence to be detected.

As used herein, the term “primer-probe detection system” refers to a method for real-time PCR. In some embodiments, the system is a Taqman based PCR system and/or a SCORPION based PCR system. In some embodiments, a primer-probe detection system comprises at least one forward primer, at least one reverse primer, and at least one oligonucleotide probe. In some embodiments, the oligonucleotide probe is detectably labeled. In some embodiments, the oligonucleotide probe comprises a detectable label and a quencher moiety.

The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e., collisional) quenching or by Forster or fluorescence resonance energy transfer (“FRET”). Quenching by FRET is generally used in TaqMan® probes while proximal quenching is used in molecular beacon and Scorpion™ type probes. Nonlimiting examples of quenchers include TAMRA, Black Hole Quencher, Deep Dark Quencher, ZEN, Iowa Black FQ, Iowa Black RQ, and DABCYL.

A “reaction-sample mixture” as used herein refers to a mixture containing amplification master mix and a sample.

As used herein, the term “sample” refers to clinical samples obtained from a patient or isolated microorganisms. In preferred embodiments, a sample is obtained from a biological source (i.e., a “biological sample”), such as tissue, bodily fluid, or microorganisms collected from a subject. Sample sources include, but are not limited to, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material). Preferred sample sources include BAL, BW, and/or throat swabs or nasal washes.

The term “sensitivity,” as used herein in reference to the methods of the present technology, is a measure of the ability of a method to detect a preselected sequence variant in a heterogeneous population of sequences. A method has a sensitivity of S % for variants of F % if, given a sample in which the preselected sequence variant is present as at least F % of the sequences in the sample, the method can detect the preselected sequence at a preselected confidence of C %, S % of the time. By way of example, a method has a sensitivity of 90% for variants of 5% if, given a sample in which the preselected variant sequence is present as at least 5% of the sequences in the sample, the method can detect the preselected sequence at a preselected confidence of 99%, 9 out of 10 times (F=5%; C=99%; S=90%). Exemplary sensitivities include at least 50, 60, 70, 80, 90, 95, 98, and 99%.

The term “specific” as used herein in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 85-95% and more preferably at least 98% sequence identity. Sequence identity can be determined using a commercially available computer program with a default setting that employs algorithms well known in the art. As used herein, sequences that have “high sequence identity” have identical nucleotides at least at about 50% of aligned nucleotide positions, preferably at least at about 60% of aligned nucleotide positions, and more preferably at least at about 75% of aligned nucleotide positions.

“Specificity,” as used herein, is a measure of the ability of a method to distinguish a truly occurring preselected sequence variant from sequencing artifacts or other closely related sequences. It is the ability to avoid false positive detections. False positive detections can arise from errors introduced into the sequence of interest during sample preparation, sequencing error, or inadvertent sequencing of closely related sequences like pseudo-genes or members of a gene family. A method has a specificity of X % if, when applied to a sample set of N_{Total} sequences, in which X_{True} sequences are truly variant and X_{Not} true are not truly variant, the method selects at least X % of the not truly variant as not variant. E.g., a method has a specificity of 90% if, when applied to a sample set of 1,000 sequences, in which 500 sequences are truly variant and 500 are not truly variant, the method selects 90% of the 500 not truly variant sequences as not variant. Exemplary specificities include at least 50, 60, 70, 80, 90, 95, 98, and 99%.

The term “stringent hybridization conditions” as used herein refers to hybridization conditions at least as stringent as the following: hybridization in 50% formamide, 5×SSC, 50 mM NaH₂PO4, pH 6.8, 0.5% SDS, 0.1 mg/mL sonicated salmon sperm DNA, and 5×Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein “TaqMan® PCR detection system” refers to a method for real-time PCR. In this method, a TaqMan® probe which hybridizes to the amplified nucleic acid segment is included in the amplification master mix. The TaqMan® probe comprises a donor and a quencher fluorophore on either end of the probe and in close enough proximity to each other so that the fluorescence of the donor is taken up by the quencher. However, when the probe hybridizes to the amplified segment, the 5′-exonuclease activity of the Taq polymerase cleaves the probe thereby allowing the donor fluorophore to emit fluorescence which can be detected.

The terms “target nucleic acid” or “target sequence” as used herein refer to a nucleic acid sequence of interest to be detected and/or quantified in the sample to be analyzed. Target nucleic acid may be composed of segments of a chromosome, a complete gene with or without intergenic sequence, segments or portions of a gene with or without intergenic sequence, or sequence of nucleic acids which probes or primers are designed. Target nucleic acids may include a wild-type sequence(s), a mutation, deletion, insertion or duplication, tandem repeat elements, a gene of interest, a region of a gene of interest or any upstream or downstream region thereof. Target nucleic acids may represent alternative sequences or alleles of a particular gene. Target nucleic acids may be derived from genomic DNA, cDNA, or RNA.

### Biological Sample Collection and Preparation

The methods and compositions of the present technology are useful in detecting pathogenic Legionella sp. by assaying for target nucleic acid sequences corresponding to the ssrA genes and 16S rRNA genes in a biological sample obtained from a subject. Samples for pathogenic Legionella sp. detection may also comprise cultures of bacterial isolates grown on appropriate media to form colonies, wherein the cultures were prepared from a biological sample obtained from a subject.

The methods disclosed herein are useful in detecting and quantifying pathogenic Legionella sp. in biological samples derived from sterile and/or non-sterile sites. “Sterile sites” include body fluids such as whole blood, plasma, cell free plasma, urine, cerebrospinal fluid, synovial fluid, pleural fluid, pericardial fluid, intraocular fluid, tissue biopsies or endrotracheal aspirates. As used herein, “cell-free plasma” refers to plasma containing less than 1% cells by volume. “Non-sterile sites” include sputum, stool, skin swabs, inguinal swabs, nasal swabs and throat swabs. In some embodiments, the biological samples comprise nasopharyngeal (NP) aspirates or swabs or nasal washes. In other embodiments, the biological samples comprise cultures of isolated bacteria grown on appropriate media to form colonies. Samples may also include bacterial isolates.

In some embodiments, the sample is transported or stored in a sterile vial containing VCM or M4 media. VCM medium comprises Hank's Balanced Salts, Bovine Serum Albumin, L-Cysteine, Gelatin, Sucrose, L-Glutamic Acid, HEPES Buffer, Vancomycin, Amphotericin B, Colistin, and optionally Phenol Red. M4 medium comprises gelatin, vancomycin, amphotericin B, and colistin.

A biological sample may be suspected of containing pathogenic Legionella sp. and/or nucleic acids of one or more pathogenic Legionella sp. In addition, a biological sample may be obtained from a subject suspected of being infected with one or more pathogenic Legionella sp. The biological sample may be contacted with an amplification master mix for use in a microfluidic/microelectronic centrifugation platform.

In some embodiments, the disclosed methods employ unprocessed biological samples thus resulting in a direct, streamlined sample-to-result process. In other embodiments, the detection methods disclosed herein will be effective if used on isolated nucleic acid (DNA or RNA) purified from a biological sample according to any methods well known to those of skill in the art. If desired, the sample may be collected or concentrated by centrifugation and the like. The cells of the sample may be subjected to lysis, such as by treatments with enzymes, heat surfactants, ultrasonication or a combination thereof. Alternatively, a biological sample may be processed using a commercially available nucleic acid extraction kit.

In some embodiments, one or more primer pairs are present in an amplification master mix that further comprises DNA polymerase, dNTPs and PCR buffer prior to contact with the biological sample. Amplification of the ssrA genes and 16S rRNA genes preferably occurs in a multiplex format. Alternatively, individual PCR reactions for each target sequence may also be used. The biological sample may be contacted with the primer pair(s) and/or with an amplification master mix to form a reaction-sample mixture in a direct amplification disc. For example, the biological sample may be contacted with the amplification master mix in a direct amplification disc such as the Direct Amplification Disc marketed by Focus Diagnostics, Inc. (Cypress, Calif., USA) as part of the SIMPLEXA Direct real-time PCR assays to work in concert with the 3M™ Integrated Cycler. A direct amplification disc is a thin, circular disc containing multiple designated regions, each of which contains a well for receiving an amplification master mix and an associated well for receiving unprocessed patient sample. The sample-reaction mixture is produced in the direct amplification disc upon or after addition of the amplification master mix and the sample.

In some embodiments, the biological sample is isolated from a subject. In some embodiments, the subject is a mammal. In some embodiments, the mammal is a bovine, equine, porcine, feline, canine, murine, simian, rat, or human. In some embodiments, the subject is a human. In particular embodiments, the subject is a human patient with one or more pneumonia-like symptoms.

### Real-Time PCR

Amplification of target nucleic acids can be detected by any of a number of methods well-known in the art such as gel electrophoresis, column chromatography, hybridization with a probe, sequencing, melting curve analysis, or “real-time” detection.

For real-time detection, primers and/or probes may be detectably labeled to allow differences in fluorescence when the primers become incorporated or when the probes are hybridized, for example, and amplified in an instrument capable of monitoring the change in fluorescence during the reaction. Real-time detection methods for nucleic acid amplification are well known and include, for example, the TaqMan® system, Scorpion™ primer system and use of intercalating dyes for double-stranded nucleic acid.

In real-time quantitative PCR, the accumulation of amplification product is measured continuously in both standard dilutions of target DNA and samples containing unknown amounts of target DNA. A standard curve is constructed by correlating initial template concentration in the standard samples with the number of PCR™ cycles (Ct) necessary to produce a specific threshold concentration of product. In the test samples, target PCR™ product accumulation is measured after the same Ct, which allows interpolation of target DNA concentration from the standard curve.

In some embodiments, amplified nucleic acids are detected by hybridization with a specific probe. Probe oligonucleotides, complementary to a portion of the amplified target sequence may be used to detect amplified fragments. In some embodiments, hybridization may be detected in real time. In an alternate embodiment, hybridization is not detected in real time. Amplified nucleic acids for each of the target sequences may be detected simultaneously (i.e., in the same reaction vessel such as multiplex PCR) or individually (i.e., in separate reaction vessels). In certain embodiments, multiple target nucleic acids are detected simultaneously, using two or more distinguishably-labeled (e.g., via different detectable moieties such as color), gene-specific oligonucleotide probes, one which hybridizes to the first target sequence and the other which hybridizes to the second target sequence.

In some embodiments, the different primer pairs are labeled with different distinguishable detectable moieties. Thus, for example, HEX and FAM fluorescent dyes may be present on different primer pairs in the multiplex PCR and associated with the resulting amplicons. In other embodiments, the forward primer is labeled with one detectable moiety, while the reverse primer is labeled with a different detectable moiety, e.g. FAM dye for a forward primer and HEX dye for a reverse primer. Use of different detectable moieties is useful for discriminating between amplified products which are of the same length or are very similar in length.

For sequence-modified nucleic acids, the target may be independently selected from the top strand or the bottom strand. Thus, all targets to be detected may comprise top strand, bottom strand, or a combination of top strand and bottom strand targets.

One general method for real-time PCR uses fluorescent probes such as the TaqMan® probes, molecular beacons, and Scorpion primer-probes. Real-time PCR quantifies the initial amount of the template with more specificity, sensitivity and reproducibility, than other forms of quantitative PCR, which detect the amount of final amplified product. Real-time PCR does not detect the size of the amplicon. The probes employed in Scorpion™ and TaqMan® technologies are based on the principle of fluorescence quenching and involve a donor fluorophore and a quenching moiety.

Real-time PCR is performed using any suitable instrument capable of detecting the accumulation of the PCR amplification product. Most commonly, the instrument is capable of detecting fluorescence from one or more fluorescent labels. For example, real-time detection on the instrument (e.g., an ABI Real-Time PCR System 7500@ sequence detector) monitors fluorescence and calculates the measure of reporter signal, or Rn value, during each PCR cycle. The threshold cycle, or Ct value, is the cycle at which fluorescence intersects the threshold value. The threshold value can be determined by the sequence detection system software or manually.

In some embodiments, the probes employed are detectably labeled and the detecting is accomplished by detecting the probe label for each amplification product. A quencher may further be associated with the detectable label which prevents detection of the label prior to amplification of the probe's target. TaqMan® probes are examples of such probes.

TaqMan® probes (Heid et al., Genome Res. 6: 986-994, 1996) use the fluorogenic 5′ exonuclease activity of Taq polymerase to measure the amount of target sequences in DNA samples. TaqMan® probes are oligonucleotides that contain a donor fluorophore usually at or near the 5′ base, and a quenching moiety typically at or near the 3′ base. The quencher moiety may be a dye such as TAMRA or may be a non-fluorescent molecule such as 4-(4-dimethylaminophenylazo) benzoic acid (DABCYL). See Tyagi et al., 16 Nature Biotechnology 49-53 (1998). When irradiated, the excited fluorescent donor transfers energy to the nearby quenching moiety by FRET rather than fluorescing. Thus, the close proximity of the donor and quencher prevents emission of donor fluorescence while the probe is intact.

TaqMan® probes are designed to anneal to an internal region of a PCR product. When the polymerase replicates a template on which a TaqMan® probe is bound, its 5′ exonuclease activity cleaves the probe. This terminates the activity of the quencher (no FRET) and the donor fluorophore starts to emit fluorescence which increases in each cycle proportional to the rate of probe cleavage. Accumulation of PCR product is detected by monitoring the increase in fluorescence of the reporter dye. If the quencher is an acceptor fluorophore, then accumulation of PCR product can be detected by monitoring the decrease in fluorescence of the acceptor fluorophore.

In certain embodiments, real-time PCR is performed using a bifunctional primer-probe detection system (e.g., Scorpion™ primers). With Scorpion primers, sequence-specific priming and PCR product detection is achieved using a single molecule. The Scorpion primer maintains a stem-loop configuration in the unhybridized state. The fluorophore is attached to the 5′ end and is quenched by a moiety coupled to the 3′ end, although in certain embodiments, this arrangement may be switched. The 3′ portion of the stem and/or loop also contains sequence that is complementary to the extension product of the primer and is linked to the 5′ end of a specific primer via a non-amplifiable monomer. After extension of the primer moiety, the specific probe sequence is able to bind to its complement within the extended amplicon, thus opening up the hairpin loop. This prevents the fluorescence from being quenched and a signal is observed. A specific target is amplified by the reverse primer and the primer portion of the Scorpion™ primer, resulting in an extension product. A fluorescent signal is generated due to the separation of the fluorophore from the quencher resulting from the binding of the probe element of the Scorpion™ primer to the extension product.

In some embodiments, the probes employed in the disclosed methods comprise or consist of short fluorescently labeled DNA sequences designed to detect sections of DNA sequence with a genetic variation such as those disclosed in French et al., Mol Cell Probes, 5(6):363-74 (2001), incorporated by reference herein in its entirety. HyBeacons® are an example of this type of probe.

In some embodiments of the method, at least one primer of each primer pair or at least one probe in the amplification reaction comprises a detectable moiety. Alternatively, the detectable moiety may be on a probe that is attached to the primer, such as with a primer-probe. In some embodiments, the detectable moiety or label is a fluorophore. Suitable fluorescent moieties include, but are not limited to the following fluorophores: 4-acetamido-4′-isothiocyanatostilbene-2,2′disulfonic acid, acridine and derivatives (acridine, acridine isothiocyanate), Alexa Fluors (Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes)), 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, BODIPY® R-6G, BOPIPY® 530/550, BODIPY® FL, Brilliant Yellow, Cal Fluor Red 610® (CFR610), coumarin and derivatives (coumarin, 7-amino-4-methylcoumarin (AMC, Coumarin 120), 7-amino-4-trifluoromethylcouluarin (Coumarin 151)), Cy2®, Cy3®, Cy3.5®, Cy5®, Cy5.5®, cyanosine, 4′,6-diaminidino-2-phenylindole (DAPI), 5′, 5″-dibromopyrogallol-sulfonephthalein (Bromopyrogallol Red), 7-diethylamino-3-(4′-isothiocyanatophenyl)-4-methylcoumarin, diethylenetriamine pentaacetate, 4,4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansyl chloride), 4-(4′-dimethylaminophenylazo)benzoic acid (DABCYL), 4-dimethylaminophenylazophenyl-4′-isothiocyanate (DABITC), Eclipse™ (Epoch Biosciences Inc.), eosin and derivatives (eosin, eosin isothiocyanate), erythrosin and derivatives (erythrosin B, erythrosin isothiocyanate), ethidium, fluorescein and derivatives (5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET), fluorescamine, IR144, IR1446, lanthamide phosphors, Malachite Green isothiocyanate, 4-methylumbelliferone, ortho cresolphthalein, nitrotyrosine, pararosaniline, Phenol Red, B-phycoerythrin, R-phycoerythrin, allophycocyanin, o-phthaldialdehyde, Oregon Green®, propidium iodide, pyrene and derivatives (pyrene, pyrene butyrate, succinimidyl 1-pyrene butyrate), QSY® 7, QSY® 9, QSY® 21, QSY® 35 (Molecular Probes), Reactive Red 4 (Cibacron® Brilliant Red 3B-A), rhodamine and derivatives (6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, tetramethyl rhodamine isothiocyanate (TRITC), riboflavin, rosolic acid, terbium chelate derivatives, Quasar 670@, and VIC®.

Suitable quenchers are selected based on the fluorescence spectrum of the particular fluorophore. Useful quenchers include, for example, the Black Hole™ quenchers BHQ-1, BHQ 2, and BHQ-3 (Biosearch Technologies, Inc.), and the ATTO-series of quenchers (ATTO 540Q, ATTO 580Q, and ATTO 612Q; Atto-Tec GmbH).

In some embodiments of the method, the reaction-sample mixture is subjected to real-time polymerase chain reaction (PCR) conditions under which each of the target nucleic acids present in the biological sample is amplified and the amplified product(s) are detected and measured. In some embodiments, the biological sample is loaded directly into a direct amplification disc without a separate, front-end specimen preparation, followed by Real-time PCR detection and differentiation of target analytes in the same disc. In certain embodiments, the amplification is performed in a Direct Amplification Disc (an 8-well disc from Focus Diagnostics, Inc.). In some embodiments, real-time PCR amplification is performed using the SIMPLEXA Direct assay in a direct amplification disc and detection is performed using an integrated thermal cycler such as the 3M™ Integrated Cycler sold by 3M (St. Paul, Minn., USA). The 3M™ Integrated Cycler can receive a Direct Amplification Disc and is capable of performing multiple assays per disc. This apparatus can heat at >5° C. per second and cool at >4° C. per second. Cycling parameters can be varied, depending on the length of the amplification products to be extended. In certain embodiments, an internal positive amplification control (IPC) can be included in the sample, utilizing oligonucleotide primers, probes and/or primer-probes.

### Alternate Methods of Detecting Target Nucleic Acids

Alternatively, detection of the target nucleic acids can occur by measuring the end-point of the reaction. In end-point detection, the amplicon(s) could be detected by first size-separating the amplicons, and then detecting the size-separated amplicons. The separation of amplicons of different sizes can be accomplished by gel electrophoresis, column chromatography, capillary electrophoresis, or other separation methods known in the art.

The detectable label can be incorporated into, associated with or conjugated to a nucleic acid. Label can be attached by spacer arms of various lengths to reduce potential steric hindrance or impact on other useful or desired properties. See, e.g., Mansfield, 9 Mol. Cell. Probes 145-156 (1995). Detectable labels can be incorporated into nucleic acids by covalent or non-covalent means, e.g., by transcription, such as by random-primer labeling using Klenow polymerase, or nick translation, or amplification, or equivalent as is known in the art. For example, a nucleotide base is conjugated to a detectable moiety, such as a fluorescent dye, and then incorporated into nucleic acids during nucleic acid synthesis or amplification.

Examples of other useful labels that aid in the detection of target nucleic acids include radioisotopes (e.g., ³²P, ³⁵S, ³H, ¹⁴C, ¹²⁵I, ¹³¹I), electron-dense reagents (e.g., gold), enzymes (e.g., horseradish peroxidase, beta-galactosidase, luciferase, alkaline phosphatase), colorimetric labels (e.g., colloidal gold), magnetic labels (e.g., Dynabeads™), biotin, dioxigenin, or haptens and proteins for which antisera or monoclonal antibodies are available. Other labels include ligands or oligonucleotides capable of forming a complex with the corresponding receptor or oligonucleotide complement, respectively. The label can be directly incorporated into the nucleic acid to be detected, or it can be attached to a probe (e.g., an oligonucleotide) or antibody that hybridizes or binds to the nucleic acid to be detected.

In other embodiments, fluorescent nucleotide analogs can be used to label nucleic acids, see, e.g., Jameson, Methods. Enzymol. 278: 363-390 (1997); Zhu, Nucl. Acids Res. 22: 3418-3422 (1994). U.S. Pat. Nos. 5,652,099 and 6,268,132 also describe nucleoside analogs for incorporation into nucleic acids, e.g., DNA and/or RNA, or oligonucleotides, via either enzymatic or chemical synthesis to produce fluorescent oligonucleotides. U.S. Pat. No. 5,135,717 describes phthalocyanine and tetrabenztriazaporphyrin reagents for use as fluorescent labels.

In some embodiments, detectably labeled probes can be used in hybridization assays including, but not limited to Northern blots, Southern blots, microarray, dot or slot blots, and in situ hybridization assays such as fluorescent in situ hybridization (FISH) to detect a target nucleic acid sequence within a biological sample. Certain embodiments may employ hybridization methods for measuring expression of a polynucleotide gene product, such as mRNA. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davis, PNAS. 80: 1194 (1983).

### Legionella Screening Assay of the Present Technology

In various embodiments of the present disclosure, primers and probes are used in the methods described herein to amplify and detect target nucleic acid sequences of pathogenic Legionella species. In certain embodiments, target nucleic acids may include the ssrA gene from all Legionella species, and the 16S rRNA gene Legionella pneumophila. In addition, primers can also be used to amplify one or more control nucleic acid sequences.

The primers and probes of the present technology are used in the methods described herein to amplify and detect a target nucleic acid comprising SEQ ID NO: 7 corresponding to the ssrA gene and a target nucleic acid comprising SEQ ID NO: 8 corresponding to the 16S rRNA gene. In one embodiment, the method involves employing primer pairs specifically directed to ssrA and 16S rRNA genes. The target nucleic acids described herein may be detected individually or in a multiplex format, utilizing individual labels for each target.

Specific primers, probes and primer-probes for amplification and detection of all or a fragment of a marker gene specific for L. pneumophila include those directed to sequences present in L. pneumophila, but absent from other Legionella species. The detection of a L. pneumophila-specific gene helps to distinguish a sample containing L. pneumophila from one that may contain another Legionella pathogenic species.

A suitable marker gene is 16S rRNA gene (see, e.g., GenBank Accession No. NC_002942.5) and is shown below.

(SEQ ID NO: 8)
AACTGAAGAGTTTGATCCTGGCTCAGATTGAACGCTGGCGGCATGCTTAA
CACATGCAAGTCGAACGGCAGCATTGTCTAGCTTGCTAGACAGATGGCGA
GTGGCGAACGGGTGAGTAACGCGTAGGAATATGCCTTGAAGAGGGGGACA
ACTTGGGGAAACTCAAGCTAATACCGCATAATGTCTGAGGACGAAAGCTG
GGGACCTTCGGGCCTGGCGCTTTAAGATTAGCCTGCGTCCGATTAGCTAG
TTGGTGGGGTAAGGGCCTACCAAGGCGACGATCGGTAGCTGGTCTGAGAG
GATGACCAGCCACACTGGAACTGAGACACGGTCCAGACTCCTACGGGAGG
CAGCAGTGGGGAATATTGGACAATGGGGGCAACCCTGATCCAGCAATGCC
GCGTGTGTGAAGAAGGCCTGAGGGTTGTAAAGCACTTTCAGTGGGGAGGA
GGGTTGATAGGTTAAGAGCTGATTAACTGGACGTTACCCACAGAAGAAGC
ACCGGCTAACTCCGTGCCAGCAGCCGCGGTAATACGGAGGGTGCGAGCGT
TAATCGGAATTACTGGGCGTAAAGGGTGCGTAGGTGGTTGATTAAGTTAT
CTGTGAAATTCCTGGGCTTAACCTGGGACGGTCAGATAATACTGGTTGAC
TCGAGTATGGGAGAGGGTAGTGGAATTTCCGGTGTAGCGGTGAAATGCGT
AGAGATCGGAAGGAACACCAGTGGCGAAGGCGGCTACCTGGCCTAATACT
GACACTGAGGCACGAAAGCGTGGGGAGCAAACAGGATTAGATACCCTGGT
AGTCCACGCTGTAAACGATGTCAACTAGCTGTTGGTTATATGAAAATAAT
TAGTGGCGCAGCAAACGCGATAAGTTGACCGCCTGGGGAGTACGGTCGCA
AGATTAAAACTCAAAGGAATTGACGGGGGCCCGCACAAGCGGTGGAGCAT
GTGGTTTAATTCGATGCAACGCGAAGAACCTTACCTACCCTTGACATACA
GTGAATTTTGCAGAGATGCATTAGTGCCTTCGGGAACACTGATACAGGTG
CTGCATGGCTGTCGTCAGCTCGTGTCGTGAGATGTTGGGTTAAGTCCCGT
AACGAGCGCAACCCTTATCCTTAGTTGCCAGCATGTGATGGTGGGGACTC
TAAGGAGACTGCCGGTGACAAACCGGAGGAAGGCGGGGATGACGTCAAGT
CATCATGGCCCTTACGGGTAGGGCTACACACGTGCTACAATGGCCGATAC
AGAGGGCGGCGAAGGGGCGACCTGGAGCAAATCCTTAAAAGTCGGTCGTA
GTCCGGATTGGAGTCTGCAACTCGACTCCATGAAGTCGGAATCGCTAGTA
ATCGCGAATCAGCATGTCGCGGTGAATACGTTCCCGGGCCTTGTACACAC
CGCCCGTCACACCATGGGAGTGGGTTGCACCAGAAGTAGATAGTCTAACC
TTCGGGGGGACGTTTACCACGGTGT

The nucleic acid sequence of the 16S rRNA amplicon generated using the methods disclosed herein is underlined.

In some embodiments, the 16S rRNA target nucleic acid comprises 5′

(SEQ ID NO: 9)
5′ TACCTACCCTTGACATACAGTGAATTTTGCAGAGATGCATTAGTGCC
TTCGGGAACACTGATACAGGTGCTGCATGGCTGTCGTCAGCTCGTGTCGT
GAGATGTTGGGTTAAGTCCCGTAACGAGCGCAACCCTTATCCTTAGTTGC
CAGCATGTGATGGTGGGGACTCTAAGGAGACTGCCGGTGACAAACCGGAG
GAAG 3′

fragment thereof.

Exemplary primer and labeled probe sequences for amplifying and detecting the 16S rRNA element include:

| | |
|---|---|
| Fwd primer | 5′ TAC CTA CCC TTG ACA TAC AGT G 3′ |
| | (SEQ ID NO: 4) |
| Rev primer | 5′ CTT CCT CCG GTT TGT CAC 3′ |
| | (SEQ ID NO: 5) |
| Probe | 5′ CCA GCA TGT GAT GGT GGG GAC TCT A 3′ |
| | (SEQ ID NO: 6) |

The ssrA gene encodes a tmRNA binding protein and is present in all Legionella sp. A nonlimiting, exemplary nucleotide sequence of ssrA is provided at GenBank accession no. AE017354.1 (bp 3213808 to 3214278) and is shown below. The nucleic acid sequence of the ssrA amplicon generated using the methods disclosed herein is underlined:

(SEQ ID NO: 7)
ATGTGGGCAGAAGTATTATCCAGCGATGCCTTTGCGCGCTTTGAGGAGGA
AGGTATTTTCAACCCCAAAACTGGACATGACTTTTTAAAATCCATTCTGG
AGGTAGGCGGCTCAAGAAAAGCAGCCGATGCTTTTGTTGAATTCAGAGGA
AGACCCGCGACGATTGATGCCTTGCTGCGCCATAACGGGATTTTATAAAA
ACAGGACTTGCGCCCCCCAATCCTCTCGGTAAAACATTTTTTGCCTTGAC
CTTGGGGTTTTCCGTAAGTCCTGAAAATATATTCGGTATGCTGGCGGGAT
TTTGCTTACATGCCGGCATTTTATGTTATAATTAAAGTGTACAGAATGGG
GGGCGACCTGGCTTCGACGTGGGTTGCAAAACCGGAAGTGCATGCCGAGA
AGGAGATCTCTCGTAAATAAGACTCAATTAAATATAAATGCAAACGATGA
AAACTTTGCTGGTGGGGAAGCTATCGCTGCCTAATAAGCACTTTAGTTAA
ACCATCACTGTGTACTGGCCAATAAACCCAGTATCCCGTTCGACCGAGCC
CGCTTATCGGTATCGAATCAACGGTCATAAGAGATAAGCTAGCGTCCTAA
TCTATCCCGGGTTATGGCGCGAAACTCAGGGAATCGCTGTGTATCATCCT
GCCCGTCGGAGGAGCCACAGTTAAATTCAAAAGACAAGGCTATGCATGTA
GAGCTAAAGGCAGAGGACTTGCGGACGCGGGTTCGATTCCCGCCGCCTCC
ACCAATTCATTATCCGATACAGTCCAATACCGGGTCTTTCCCAAATACCT
GAATCTTCTACACATCTTGTTTATTCCAAACAAACATGATCAAATCACCT
CTTTTTGAGGTATGTATGGACTTAGCAGTTGAAGATACTACAGCATGGTC
GGAAGCTATTTTTGGTTCAGTTGCTTTAGGGGATAAACGACTTACTCGTC
GGTTAATTCAAATAGGCAAACAATTATCATCGACGCCTGGTGGTTCTCTT
TCAGGAAGTTGTGGAGGGCAGGATGCGCTTATAGAAGGTAGTTATCGTTT
TTTACGAAACAAACGAGTCACAGCGAATCAAATTGCAGAGGGTGGTT

In some embodiments, the ssrA target nucleic acid comprises 5′

(SEQ ID NO: 10)
5′ TCGACGTGGGTTGCAAAACCGGAAGTGCATGCCGAGAAGGAGATCTC
TCGTAAATAAGACTCAATTAAATATAAATGCAAACGATGAAAACTTTGCT
GGTGGGGAAGCTATCGCTGCCTAATAAGCACTTTAGTTAAACCATCACTG
TGTACTGGCCAATAAACCCAGTATCCCGTTCGACCGAGCCCGCTTATCGG
TATCGAATCAACGGTCATA 3′

or a fragment thereof.

Exemplary primer and labeled probe sequences for amplifying and detecting the ssrA target sequence:

| | |
|---|---|
| Fwd primer | 5′ TCG ACG TGG GTT GCR AAA CG 3′ |
| | (SEQ ID NO: 1) |
| | 5′ TCG ACG TGG GTT GCA AAA CG 3′ |
| | (SEQ ID NO: 11) |
| | 5′ TCG ACG TGG GTT GCT AAA CG 3′ |
| | (SEQ ID NO: 12) |
| | 5′ TCG ACG TGG GTT GCC AAA CG 3′ |
| | (SEQ ID NO: 13) |
| | 5′ TCG ACG TGG GTT GCG AAA CG 3′ |
| | (SEQ ID NO: 14) |
| Rev primer | 5′ TAT GAC CGT TGA TTC GAT ACC3′ |
| | (SEQ ID NO: 2) |
| Probe | 5′ TAA ATA TAA ATG CAA ACG ATG AAA |
| | ACT TTG C 3′ |
| | (SEQ ID NO: 3) |

In some embodiments, the ssrA detection assay does not detect non-Legionella species of bacteria. For example, in some embodiments, the ssrA assay comprises PCR primers and probes that are specific for Legionella sp. and do not amplify nucleic acids derived from one or more of the following: Bacillus cereus, Chlamydophila pneumoniae, Haemophilus influenzae, Klebsiella pneumonia, RSV B, Mycoplasma pneumonia, Streptococcus pneumoniae, Staphylococcus aureus, Moraxella catarrhalis Influenza A, Influenza B, Pseudomonas sp., and Enterobacter sp.

Qualitative detection and differentiation of Legionella sp. and L. pneumophila using the disclosed method may utilize primer pairs, labeled probes and real-time PCR for amplification and detection of the ssrA and 16S rRNA genes on a direct amplification disc with an integrated cycler system. With this method, target genomic DNA is specifically amplified and simultaneously detected by fluorescent-labeled probes in the same reaction.

In some embodiments, one or more of the primers and/or probes used herein is a degenerate primer or probe, meaning a mix of oligonucleotide sequences in which some positions contain a number of possible bases, giving a population of primers with similar sequences that cover all possible nucleotide combinations for a given protein sequence. A degenerate nucleotide is designated by an R. A non-limiting example of a degenerate primer is SEQ ID NO: 1.

In some embodiments, a positive control comprising nucleic acid derived from a L. pneumophila stock organism is used for the screening assay. For example, suitable control DNA is available from Microbiologics (Cat. #0211P). In some embodiments, the positive control is diluted in a buffer, such as TE buffer or water. In some embodiments, the dilution is about 1:1,000, 1:10,000, 1:100,000, 1:1,000,000, or 1:10,000,000 in the buffer.

In some embodiments, an amplification or extraction control comprising exogenous nucleic acid is used for the screening assay. In some embodiments, the amplification or extraction control sample does not comprise nucleic acids derived from Legionella sp. For example, suitable control DNA is available from Diasorin (Cat. #151599). In some embodiments, the amplification or extraction control is diluted in a buffer, such as TE buffer or water. In some embodiments, the dilution is about 1:1,000, 1:10,000, 1:100,000, 1:1,000,000, or 1:10,000,000 in the buffer. In some embodiments, the amplification or extraction control sample is admixed with the biological sample prior to amplification of the target nucleic acids.

In some embodiments, the control nucleic acid sequence comprises

(SEQ ID NO: 13)
5′ TAACCCCGCGATAAAGACAGAAGATTATGCATACGAGATCAAAGGA
GCCGGCCTTTTCTCTAGAGATCTCTTATTTTCCTTGAAGTCACCTGTTT
ATGTTAAAGCAGGTGAGCAGGTATACATTCAGTACGATCTGAACAAAAG
CAATGCAGAACTTGCTCTCGACTATGGTTTTGTGGAATCAAACCCTAAA
CGGAACTCATATACTTTAACAATAGAGATACCAGAATCAGACCCATTCT
TTGGGGATAAGTTGGATATTGCTGAGAGTAACAAGATGGGTGAGACCGG
ATACTTTGACATAGTAGACGGCCAGACTCTTCCCGCTGGTATGCTTCAG
TACCTTCGGCTTGTGGCTCTTGGCGGTCCAGATGCTTTCTTATTAGAAT
CTATCTTCAATAACACCATATGGGGTCATCTTGAATTGCCTGTAAGTCG
TACAAACGAGGAACTCATATGCCGTGTTGTCAGAGATGCCTGCAAATCT
GCTCTGTCTGGTTTTGATACGACCATTGAAGAGGATGAGAAGCTTCTGG
ACAAAGGAAAGCTTGAGCCTAGGTTGGAAATGGCTCTCAAG 3′,

and a forward primer comprising 5′ GCTTCAGTACCTTCGGCTTG 3′ (SEQ ID NO: 17), a reverse primer comprising 5′ TTGCAGGCATCTCTGACAAC 3′ (SEQ ID NO: 18) and a detectably labelled nucleic acid probe comprising 5′ TGGCTCTTGGCGGTCCAGATG 3′ (SEQ ID NO: 19) are used to amplify the control nucleic acid sequence.

In some embodiments, the control target nucleic acid comprises

(SEQ ID NO: 20)
5′ GCTTCAGTACCTTCGGCTTGTGGCTCTTGGCGGTCCAGATGCTTTCT
TATTAGAATCTATCTTCAATAACACCATATGGGGTCATCTTGAATTGCCT
GTAAGTCGTACAAACGAGGAACTCATATGCCGTGTTGTCAGAGATGCCTG
CAA 3′.

In some embodiments, a negative control that does not does not comprise nucleic acids derived from Legionella sp. is used for the screening assay. Non-limiting examples of a suitable negative control include nuclease-free water, sterile nuclease-free water, and TE buffer.

Accordingly, in some aspects, provided herein are methods for detecting the presence of at least one Legionella species in a biological sample, the methods comprising, consisting of, or consisting essentially of: (a) providing a first primer pair suitable for amplifying an ssrA target nucleic acid; providing a second primer pair suitable for amplifying a 16S rRNA target nucleic acid; amplifying the ssrA target nucleic acid and the 16S rRNA target nucleic acid, if present; and detecting one or more amplification products produced in step (c); wherein the presence of the ssrA target nucleic acid identifies the presence of at least one Legionella species, and the presence of the 16S rRNA target nucleic acid identifies the presence of Legionella pneumophila.

### Treatment for Legionella Infection

Disclosed herein are methods for determining whether a patient exhibiting pneumonia-like symptoms will benefit from treatment with therapeutic agents that inhibit Legionella sp. and/or L. pneumophila.

Accordingly, provided herein are methods for selecting a subject exhibiting pneumonia-like symptoms for treatment with a therapeutic agent that inhibits Legionella pneumophila, the methods comprising, consisting of, or consisting essentially of: (a) contacting a sample isolated from the subject with a first primer pair suitable for amplifying an ssrA target nucleic acid; (b) contacting the sample with a second primer pair suitable for amplifying a 16S rRNA target nucleic acid; (c) amplifying the ssrA target nucleic acid and the 16S rRNA target nucleic acid, if present; and (d) detecting one or more amplification products produced in step (c); and (e) selecting the subject for treatment with a therapeutic agent that inhibits Legionella pneumophila if an amplification product for the 16S rRNA target nucleic acid is detected.

Also provided herein are methods of treating a subject with a Legionella pneumophila infection, the method comprising, consisting of, or consisting essentially of administering a therapeutic agent that inhibits Legionella pneumophila to a subject selected a method comprising, consisting of, or consisting essentially of: (a) contacting a sample isolated from the subject with a first primer pair suitable for amplifying an ssrA target nucleic acid; (b) contacting the sample with a second primer pair suitable for amplifying a 16S rRNA target nucleic acid; (c) amplifying the ssrA target nucleic acid and the 16S rRNA target nucleic acid, if present; and (d) detecting one or more amplification products produced in step (c); and (e) selecting the subject for treatment with a therapeutic agent that inhibits Legionella pneumophila if an amplification product for the 16S rRNA target nucleic acid is detected.

In some embodiments of the methods provided herein, the first primer pair comprises at least one degenerate primer. In some embodiments, the first primer pair comprises a first forward primer comprising 5′ TCGACGTGGGTTGCRAAACG 3′ (SEQ ID NO: 1) or a complement thereof. The method of any one of the previous claims, wherein the first primer pair comprises a first reverse primer comprising 5′ TATGACCGTTGATTCGATACC 3′(SEQ ID NO: 2) or a complement thereof. In some embodiments, the second primer pair comprises at least one degenerate primer. In some embodiments, the second primer pair comprises a second forward primer comprising 5′ TACCTACCCTTGACATACAGTG 3′ (SEQ ID NO: 4) or a complement thereof. In some embodiments, second primer pair comprises a second reverse primer comprising 5′ CTTCCTCCGGTTTGTCAC 3′ (SEQ ID NO: 5) or a complement thereof.

In some embodiments, the methods further comprise contacting the biological sample with one or more oligonucleotide probes capable of specifically hybridizing to an amplification product or a complement thereof. In some embodiments, the oligonucleotide probe is detectably labeled. In some embodiments, the detectable label is a fluorescent label. In some embodiments, the fluorescent label is selected from the group consisting of fluorescein, Cy3, Cy5, Cy5.5 tetrachloro-6-car-boxyfluorescein, 2,7-dimethoxy-4,5-dichloro-6-carboxy-fluorescein, Yakima Yellow, Texas Red, TYE 563, ROX, TEX 615, TYE 665, TYE 705, and hexacholoro-6-carboxyfluorescein. In some embodiments, the oligonucleotide probe further comprises at least one quencher. In some embodiments, the quencher is selected from the group consisting of TAMRA, Black Hole Quencher, Deep Dark Quencher, ZEN, Iowa Black FQ, Iowa Black RQ, and DABCYL. In some embodiments, the oligonucleotide probe specifically hybridizes to an ssrA amplification product and wherein the oligonucleotide probe comprises 5′ TAAATATAAATGCAAACGATGAAAACTTTGC 3′(SEQ ID NO: 3) or a complement thereof. In some embodiments, the oligonucleotide probe specifically hybridizes to a 16S rRNA amplification product and wherein the oligonucleotide probe comprises 5′ CCAGCATGTGATGGTGGGGACTCTA 3′(SEQ ID NO: 6) or a complement thereof.

In some embodiments, the methods further comprise admixing exogenous control DNA with the biological sample. In some embodiments, the methods further comprise contacting the biological sample with a third primer pair suitable for amplification of an exogenous control target nucleic acid and amplifying the exogenous control target nucleic acid. In some embodiments, the exogenous control target nucleic acid comprises SEQ ID NO: 20. In some embodiments, the third primer pair consists of a third forward primer comprising 5′ GCTTCAGTACCTTCGGCTTG 3′ (SEQ ID NO: 17) and a third reverse primer comprising 5′ TTGCAGGCATCTCTGACAAC 3′ (SEQ ID NO: 18). In some embodiments, the methods further comprise contacting the biological sample with a third oligonucleotide probe, wherein the third oligonucleotide probe is detectably labeled and comprises 5′ TGGCTCTTGGCGGTCCAGATG 3′ (SEQ ID NO: 19).

In some embodiments of the methods provided herein, the real-time PCR amplification is performed in a direct amplification disc in concert with an integrated thermal cycler.

In some embodiments of the methods provided herein, the biological sample is a bronchoalveolar lavage sample, a bronchial wash sample, a sputum sample, a nasopharyngeal (NP) aspirate or wash sample, a nasal swab, or a bacterial isolate.

Examples of therapeutic agents that inhibit Legionella sp. and/or L. pneumophila include fluoroquinolones, carbapenems, trimethoprim-sulfamethoxazole (e.g., Bactrim, Septra), and L. pneumophila-specific antibodies. In some embodiments, the fluoroquinolones are selected from the group consisting of ciprofloxacin, gemifloxacin, levofloxacin, norfloxacin, ofloxacin, rovafloxacin, gatifloxacin, grepafloxacin, temafloxacin, lomefloxacin, sparfloxacin, enoxacin, and moxifloxacin. In certain embodiments, the carbapenems are selected from the group consisting of imipenem, meropenem, ertapenem, doripenem, panipenem, biapenem, razupenem (PZ-601), tebipenem, lenapenem, tomopenem, and thienpenem (Thienamycin).

Examples of therapeutic agents that inhibit Legionella sp. and/or L. pneumophila include whole-cell (wP) Legionella sp. and/or L. pneumophila vaccine, acellular Legionella sp. and/or L. pneumophila vaccine, trimethoprim-sulfamethoxazole (e.g., Bactrim, Septra), telithromycin and macrolide-antibiotics. In some embodiments, the macrolide-antibiotics are selected from the group consisting of azithromycin (Zithromax), clarithromycin (Biaxin), erythromycin (E-Mycin, Eryc, Ery-Tab, PCE, Pediazole, Ilosone), and roxithromycin.

Examples of additional therapeutic agents that inhibit Legionella sp. and/or L. pneumophila include trimethoprim-sulfamethoxazole (e.g., Bactrim, Septra), ciprofloxacin, gemifloxacin, levofloxacin, norfloxacin, ofloxacin, rovafloxacin, gatifloxacin, grepafloxacin, temafloxacin, lomefloxacin, sparfloxacin, enoxacin, and moxifloxacin.

Pneumonia-like symptoms include but are not limited to chest pain, confusion, changes in mental awareness, cough, phlegm, fatigue, fever, sweating, shaking, chills, lower than normal body temperature, nausea, vomiting, diarrhea, shortness of breath, inflammation of the lungs, and fluid in the lungs.

In some embodiments, the subject is a mammal. In some embodiments, the mammal is a bovine, equine, porcine, feline, canine, murine, simian, rat, or human. In some embodiments, the subject is a human. In particular embodiments, the subject is a human patient with one or more pneumonia-like symptoms.

### Interpretation of Results

Upon subjecting the sample-reaction mixtures to real-time PCR, and detecting and measuring the fluorescence signals associated with the amplified ssrA, 16S rRNA, and control target sequences, the methods of the present technology further provide an algorithm for determining the presence of one or more related pathogenic Legionella sp., which provides the final results by matching cycle threshold (Ct) from the amplified target nucleic acid sequences.

In some embodiments, a positive Ct is a Ct less than or equal to about 35, about 36, about 37, about 38, about 39 or about 40 for a reaction comprising 40 cycles. In some embodiments, a negative Ct is a Ct greater than about 35, about 36, about 37, or about 38, about 39, or a Ct of about 40 for a reaction comprising 40 cycles. In some embodiments, a positive Ct is a Ct less than or equal to about 40, about 41, about 42, about 43, about 44 or about 45 for a reaction comprising 45 cycles. In some embodiments, a negative Ct is a Ct greater than about 40, about 41, about 42, about 43, or about 44, or a Ct of about 45 for a reaction comprising 45 cycles. In some embodiments, a positive Ct is a Ct less than or equal to about 45, about 46, about 47, about 48, about 49 or about 50 for a reaction comprising 50 cycles. In some embodiments, a negative Ct is a Ct greater than about 45, about 46, about 47, about 48, or about 49, or a Ct of about 50 for a reaction comprising 50 cycles.

The Legionella sp. algorithm dictates:

### Negative Control

| If the result is . . . | Then . . . |
|---|---|
| Positive (e.g., a Ct value < 40) | The control is invalid. |
| and with a valid | This indicates possible |
| amplification curve | contamination of prepared samples. |
| Negative (e.g., a Ct value of | The control is valid. |
| undetermined and IPC | |
| Ct is in range) | |

### Positive Control

| If the result is . . . | Then . . . |
|---|---|
| Negative or out of range | Possible inhibition or improper formulation |
| | of the mastermix. The control is invalid. |
| Positive and within range | The control is valid. |

Accordingly, the presence or absence of pathogenic Legionella sp. in a biological sample can be determined based on the following scenarios:

| If the result is . . . | Then . . . |
|---|---|
| Positive for ssrA (e.g., Ct < 37) | The sample comprises a |
| and negative | Legionella species other than |
| for 16S rRNA (e.g., Ct > 37) | L. pneumophila |
| Positive for ssrA (e.g., Ct < 37) | The sample comprises |
| and positive | L. pneumophila |
| 16S rRNA (e.g., Ct < 37) | |
| Negative for ssrA (e.g., Ct > 37) | The sample does not comprise |
| and negative | a Legionella species |
| for 16S rRNA (e.g., Ct > 37) | |

### Kits and Compositions

The present disclosure also provides kits for detecting target nucleic acid sequences corresponding to pathogenic Legionella species.

Accordingly, provided herein are kits for detecting the presence of at least one Legionella species in a biological sample, the kits comprising, consisting of, or consisting essentially of: (a) a first primer pair that amplifies an ssrA target nucleic acid; (b) a second primer pair that amplifies a 16S rRNA target nucleic acid; (c) a first oligonucleotide probe capable of specifically hybridizing to a segment of the ssrA target nucleic acid; and (d) a second oligonucleotide probe capable of specifically hybridizing to a segment of the 16S rRNA target nucleic acid; wherein the first oligonucleotide probe and the second oligonucleotide probe are detectably labeled.

In some embodiments of the kits provided herein, the kits further comprise a third primer pair that that amplifies a control target nucleic acid. In some embodiments, the first primer pair is capable of specifically hybridizing to a ssrA target nucleic acid comprising nucleotides that are at least 85-95% identical to SEQ ID NO: 7, or a complement thereof. In some embodiments, the second primer pair is capable of specifically hybridizing to a 16S rRNA target nucleic acid comprising nucleotides that are at least 85-95% identical to SEQ ID NO: 8, or a complement thereof.

In some embodiments of the kits provided herein, the first primer pair comprises at least one degenerate primer. In some embodiments, the first primer pair comprises a first forward primer comprising 5′ TCGACGTGGGTTGCRAAACG 3′ (SEQ ID NO: 1) or a complement thereof. In some embodiments, the first primer pair comprises a first reverse primer comprising 5′ TATGACCGTTGATTCGATACC 3′(SEQ ID NO: 2) or a complement thereof. In some embodiments, the second primer pair comprises at least one degenerate primer. In some embodiments, the second primer pair comprises a second forward primer comprising 5′ TACCTACCCTTGACATACAGTG 3′ (SEQ ID NO: 4) or a complement thereof. In some embodiments, the second primer pair comprises a second reverse primer comprising 5′ CTTCCTCCGGTTTGTCAC 3′ (SEQ ID NO: 5) or a complement thereof. In some embodiments, the first nucleic acid probe comprises 5′ TAAATATAAATGCAAACGATGAAAACTTTGC 3′(SEQ ID NO: 3), or a complement thereof. In some embodiments, the second nucleic acid probe comprises 5′ CAACCAGCCGCTGCTGACGGTC 3′ (SEQ ID NO: 9), or a complement thereof.

In some embodiments of the kits provided herein, the detectable label is a fluorescent label. In some embodiments, the fluorescent label is selected from the group consisting of fluorescein, Cy3, Cy5, Cy5.5 tetrachloro-6-car-boxyfluorescein, 2,7-dimethoxy-4,5-dichloro-6-carboxy-fluorescein, Yakima Yellow, Texas Red, TYE 563, ROX, TEX 615, TYE 665, TYE 705, and hexacholoro-6-carboxyfluorescein. In some embodiments, at least one oligonucleotide probe further comprises at least one quencher. In some embodiments, the oligonucleotide probe comprises two quenchers. In some embodiments, the quencher is selected from the group consisting of TAMRA, Black Hole Quencher, Deep Dark Quencher, ZEN, Iowa Black FQ, Iowa Black RQ, and DABCYL.

Kits of the present technology comprise at least two oligonucleotides which may serve as primers or primer-probes for amplifying ssrA and 16S rRNA target nucleic acid sequences to determine the presence of pathogenic Legionella sp. in a biological sample.

In some embodiments, the kit comprises liquid medium containing the at least one target-specific nucleic acid probe in a concentration of 250 nM or less. With such a kit, the probes are provided in the required amount to perform reliable multiplex detection reactions according to the present technology. In some embodiments, the target-specific nucleic acid probes are detectably labeled.

In some embodiments, the kits further comprise buffers, enzymes having polymerase activity, enzymes having polymerase activity and lacking 5′→3′ exonuclease activity or both 5′→3′ and 3′→5′ exonuclease activity, enzyme cofactors such as magnesium or manganese, salts, chain extension nucleotides such as deoxynucleoside triphosphates (dNTPs), modified dNTPs, nuclease-resistant dNTPs or labeled dNTPs, necessary to carry out an assay or reaction, such as amplification and/or detection of target nucleic acid sequences corresponding to pathogenic Legionella species.

In one embodiment, the kits of the present technology further comprise a positive control nucleic acid sequence and a negative control nucleic acid sequence to ensure the integrity of the assay during experimental runs. A kit may further contain a means for comparing the copy number of one or more of ssrA and 16S rRNA in a biological sample with a reference nucleic acid sample (e.g., a sample having a known copy number for one or more of ssrA and 16S rRNA). The kit may also comprise instructions for use, software for automated analysis, containers, packages such as packaging intended for commercial sale and the like.

The kit may further comprise one or more of: wash buffers and/or reagents, hybridization buffers and/or reagents, labeling buffers and/or reagents, and detection means. The buffers and/or reagents are usually optimized for the particular amplification/detection technique for which the kit is intended. Protocols for using these buffers and reagents for performing different steps of the procedure may also be included in the kit.

The kit additionally may comprise an assay definition scan card and/or instructions such as printed or electronic instructions for using the oligonucleotides in an assay. In some embodiments, a kit comprises an amplification reaction mixture or an amplification master mix. Reagents included in the kit may be contained in one or more containers, such as a vial.

Primers, probes, and/or primer-probes specific for amplification and detection of DNA internal control may be included in the amplification master mix as the target primer pairs to monitor potential PCR inhibition. Reagents necessary for amplification and detection of targets and internal control may be formulated as an all-in-one amplification master mix, which may be provided as single reaction aliquots in a kit.

In one aspect, provided herein is a composition comprising, consisting of, or consisting essentially of a detectably labeled oligonucleotide probe comprising 5′ TAAATATAAATGCAAACGATGAAAACTTTGC 3′ (SEQ ID NO: 3). In some embodiments, the detectable label is a fluorescent label. In some embodiments, the fluorescent label is selected from the group consisting of fluorescein, Cy3, Cy5, Cy5.5 tetrachloro-6-car-boxyfluorescein, 2,7-dimethoxy-4,5-dichloro-6-carboxy-fluorescein, Yakima Yellow, Texas Red, TYE 563, ROX, TEX 615, TYE 665, TYE 705, and hexacholoro-6-carboxyfluorescein. In some embodiments, the oligonucleotide probe further comprises at least one quencher. In some embodiments, the quencher is selected from the group consisting of TAMRA, Black Hole Quencher, Deep Dark Quencher, ZEN, Iowa Black FQ, Iowa Black RQ, and DABCYL.

In another aspect, provided herein is a composition comprising a detectably labeled oligonucleotide probe comprising 5′ TAAATATAAATGCAAACGATGAAAACTTTGC 3′ (SEQ ID NO: 3). In some embodiments, the detectable label is a fluorescent label. In some embodiments, the fluorescent label is selected from the group consisting of fluorescein, Cy3, Cy5, Cy5.5 tetrachloro-6-car-boxyfluorescein, 2,7-dimethoxy-4,5-dichloro-6-carboxy-fluorescein, Yakima Yellow, Texas Red, TYE 563, ROX, TEX 615, TYE 665, TYE 705, and hexacholoro-6-carboxyfluorescein. In some embodiments, the oligonucleotide probe further comprises at least one quencher. In some embodiments, the quencher is selected from the group consisting of TAMRA, Black Hole Quencher, Deep Dark Quencher, ZEN, Iowa Black FQ, Iowa Black RQ, and DABCYL.

### EXAMPLES

### Example 1: Detection of Pathogenic Legionella Species Using Real-Time PCR

Bronchoalveolar lavage samples are collected from patients. An internal positive control DNA target is added to the external lysis buffer prior to extraction. DNA is extracted using the MagNA Pure 96 instrument, using the “DNA/Viral NA SV 2.0” kit. Elution volume is set to 50.

A Legionella PCR mastermix is created comprising the following reagents and stored at −10° C. to −90° C.:

| | μL per | Unit of measure | | Final Concentration |
|---|---|---|---|---|
| | reaction | (1000 rxns) | | per reaction |
| Sterile Nuclease- | 1.45 | 1.45 | mL | |
| Free Water | | | | |
| ssrA forward | 0.125 | 125 | μL | 500 nM |
| primer (100 μM) | | | | |
| ssrA reverse primer | 0.125 | 125 | μL | 500 nM |
| (100 μM) | | | | |
| ssrA probe | 0.05 | 25 | μL | 100 nM |
| (100 μM) | | | | |
| 16S forward primer | 0.125 | 125 | μL | 500 nM |
| (100 μM) | | | | |
| 16S reverse primer | 0.125 | 125 | μL | 500 nM |
| (100 μM) | | | | |
| 16S probe (100 μM) | 0.05 | 25 | μL | 100 nM |
| Focus DNA Control | | | | |
| Primer Pair (SC | 0.5 | 500 | μL | 1X |
| #151600) | | | | |
| TaqPath qPCR | 12.5 | 12.5 | mL | 1X |
| Mixes, CG | | | | |
| Total | 15 μL | 15 | mL | |

Real time PCR is performed using the following conditions: i) sample pre-heat at 500 C, 120 seconds, 1 cycle ii) polymerase activation at 950 C, 10 minutes, 1 cycle, and iii) Denaturation at 95° C., 15 seconds and annealing at 60° C., 35 seconds for 40 cycles.

Target genomic DNA is specifically amplified and simultaneously detected by fluorescent-labeled probes in the same reaction. The Ct is detected an the results are analyzed according to the following algorithm:

| If the result is . . . | Then . . . |
|---|---|
| Positive for ssrA (Ct < 37) and | The sample comprises a |
| negative for | Legionella species other than |
| 16S rRNA (Ct > 37) | L. pneumophila |
| Positive for ssrA (Ct < 37) and | The sample comprises |
| positive 16S | L. pneumophila |
| rRNA (Ct < 37) | |
| Negative for ssrA (Ct > 37) and | The sample does not |
| negative for | comprise a Legionella |
| 16S rRNA (Ct > 37) | species |

### Example 2: Cross-Reactivity of the Legionella Multiplex Assay

For cross-reactivity assays, control nasal swab specimens will be spiked with one of the test organisms listed below (n=5 for each organism).

| | |
|---|---|
| Bacillus cereus | Chlamydophila pneumoniae |
| Haemophilus influenzae | Klebsiella pneumonia |
| RSV B | Mycoplasma pneumonia |
| Streptococcus pneumoniae | Staphylococcus aureus |
| Moraxella catarrhalis | Influenza A |
| Influenza B | Pseudomonas sp. |
| Enterobacter sp. | |

The Legionella multiplex assay will be performed on each sample. In each case, a Ct value≤40 is interpreted as a positive result for Legionella cross-reactivity, a Ct value of ≤40 is interpreted as a positive result for Legionella cross-reactivity, and a Ct value of ≤40 is interpreted as a positive result for Legionella cross-reactivity.

It is anticipated that no cross-reactivity will be observed for any of the above microbial species.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

The terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for detecting the presence of at least one Legionella species in a biological sample, the method comprising:
(a) providing a first primer pair suitable for amplifying an ssrA target nucleic acid;
(b) providing a second primer pair suitable for amplifying a 16S rRNA target nucleic acid;
(c) amplifying the ssrA target nucleic acid and the 16S rRNA target nucleic acid, if present; and
(d) detecting one or more amplification products produced in step (c);
wherein the presence of the ssrA target nucleic acid identifies the presence of at least one Legionella species, and the presence of the 16S rRNA target nucleic acid identifies the presence of Legionella pneumophila.

2. The method of claim 1, wherein the first primer pair comprises at least one degenerate primer.

3. The method of claim 1 or 2, wherein the first primer pair comprises a first forward primer comprising 5′ TCGACGTGGGTTGCRAAACG 3′ (SEQ ID NO: 1) or a complement thereof.

4. The method of any one of the previous claims, wherein the first primer pair comprises a first reverse primer comprising 5′ TATGACCGTTGATTCGATACC 3′(SEQ ID NO: 2) or a complement thereof.

5. The method of any one of the previous claims, wherein the second primer pair comprises at least one degenerate primer.

6. The method of any one of the previous claims, wherein the second primer pair comprises a second forward primer comprising 5′ TACCTACCCTTGACATACAGTG 3′ (SEQ ID NO: 4) or a complement thereof.

7. The method of any one of the previous claims, wherein the second primer pair comprises a second reverse primer comprising 5′ CTTCCTCCGGTTTGTCAC 3′ (SEQ ID NO: 5) or a complement thereof.

8. The method of any one of the previous claims, further comprising contacting the biological sample with one or more oligonucleotide probes capable of specifically hybridizing to an amplification product or a complement thereof.

9. The method of claim 8, wherein the oligonucleotide probe is detectably labeled.

10. The method of claim 9, wherein the detectable label is a fluorescent label.

11. The method of claim 10, wherein the fluorescent label is selected from the group consisting of fluorescein, Cy3, Cy5, Cy5.5 tetrachloro-6-car-boxyfluorescein, 2,7-dimethoxy-4,5-dichloro-6-carboxy-fluorescein, Yakima Yellow, Texas Red, TYE 563, ROX, TEX 615, TYE 665, TYE 705, and hexacholoro-6-carboxyfluorescein.

12. The method of claim 10, wherein the oligonucleotide probe further comprises at least one quencher.

13. The method of claim 12, wherein the quencher is selected from the group consisting of TAMRA, Black Hole Quencher, Deep Dark Quencher, ZEN, Iowa Black FQ, Iowa Black RQ, and DABCYL.

14. The method of any one of claims 8-13, wherein the oligonucleotide probe specifically hybridizes to an ssrA amplification product and wherein the oligonucleotide probe comprises 5′ TAAATATAAATGCAAACGATGAAAACTTTGC 3′(SEQ ID NO: 3) or a complement thereof.

15. The method of any one of claims 8-14, wherein the oligonucleotide probe specifically hybridizes to a 16S rRNA amplification product and wherein the oligonucleotide probe comprises 5′ CCAGCATGTGATGGTGGGGACTCTA 3′(SEQ ID NO: 6) or a complement thereof.

16. The method of any one of claims 1-15, further comprising admixing exogenous control DNA with the biological sample.

17. The method of claim 16, further comprising contacting the biological sample with a third primer pair suitable for amplification of an exogenous control target nucleic acid and amplifying the exogenous control target nucleic acid.

18. The method of claim 17, wherein the exogenous control target nucleic acid comprises SEQ ID NO: 20.

19. The method of claim 18, wherein the third primer pair consists of a third forward primer comprising 5′ GCTTCAGTACCTTCGGCTTG 3′ (SEQ ID NO: 17) and a third reverse primer comprising 5′ TTGCAGGCATCTCTGACAAC 3′ (SEQ ID NO: 18).

20. The method of claim 17 or 18, further comprising contacting the biological sample with a third oligonucleotide probe, wherein the third oligonucleotide probe is detectably labeled and comprises 5′ TGGCTCTTGGCGGTCCAGATG 3′ (SEQ ID NO: 19).

21. The method of any one of claims 1-20, wherein real-time PCR amplification is performed in a direct amplification disc in concert with an integrated thermal cycler.

22. The method of any one of claims 1-21, wherein the biological sample is a bronchoalveolar lavage sample, a bronchial wash sample, a sputum sample, a nasopharyngeal (NP) aspirate or wash sample, a nasal swab, or a bacterial isolate.

23. A kit for detecting the presence of at least one Legionella species in a biological sample comprising:
(a) a first primer pair that amplifies an ssrA target nucleic acid;
(b) a second primer pair that amplifies a 16S rRNA target nucleic acid;
(c) a first oligonucleotide probe capable of specifically hybridizing to a segment of the ssrA target nucleic acid; and
(d) a second oligonucleotide probe capable of specifically hybridizing to a segment of the 16S rRNA target nucleic acid;
wherein the first oligonucleotide probe and the second oligonucleotide probe are detectably labeled.

24. The kit of claim 23, further comprising a third primer pair that that amplifies a control target nucleic acid.

25. The kit of claim 23 or 24, wherein the first primer pair is capable of specifically hybridizing to a ssrA target nucleic acid comprising nucleotides that are at least 85-95% identical to SEQ ID NO: 1, or a complement thereof.

26. The kit of any one of claims 23-25, wherein the second primer pair is capable of specifically hybridizing to a 16S rRNA target nucleic acid comprising nucleotides that are at least 85-95% identical to SEQ ID NO: 2, or a complement thereof.

27. The kit of any one of claims 23-26, wherein the first primer pair comprises at least one degenerate primer.

28. The kit of any one of claims 23-27, wherein the first primer pair comprises a first forward primer comprising 5′ TCGACGTGGGTTGCRAAACG 3′ (SEQ ID NO: 1) or a complement thereof.

29. The kit of any one of claims 23-28, wherein the first primer pair comprises a first reverse primer comprising 5′ TATGACCGTTGATTCGATACC 3′(SEQ ID NO: 2) or a complement thereof.

30. The kit of any one of claims 23-29, wherein the second primer pair comprises at least one degenerate primer.

31. The kit of any one of claims 23-30, wherein the second primer pair comprises a second forward primer comprising 5′ TACCTACCCTTGACATACAGTG 3′ (SEQ ID NO:
4) or a complement thereof.

32. The kit of any one of claims 23-31, wherein the second primer pair comprises a second reverse primer comprising 5′ CTTCCTCCGGTTTGTCAC 3′ (SEQ ID NO: 5) or a complement thereof.

33. The kit of any one of claims 23-32, wherein the first nucleic acid probe comprises 5′ TAAATATAAATGCAAACGATGAAAACTTTGC 3′(SEQ ID NO: 3), or a complement thereof.

34. The kit of any one of claims 23-33, wherein the second nucleic acid probe comprises 5′ CAACCAGCCGCTGCTGACGGTC 3′ (SEQ ID NO: 9), or a complement thereof.

35. The kit of any one of claims 23-34, wherein the detectable label is a fluorescent label.

36. The kit of claim 35, wherein the fluorescent label is selected from the group consisting of fluorescein, Cy3, Cy5, Cy5.5 tetrachloro-6-car-boxyfluorescein, 2,7-dimethoxy-4,5-dichloro-6-carboxy-fluorescein, Yakima Yellow, Texas Red, TYE 563, ROX, TEX 615, TYE 665, TYE 705, and hexacholoro-6-carboxyfluorescein.

37. The kit of any one of claims 23-36, wherein at least one oligonucleotide probe further comprises at least one quencher.

38. The kit of claim 37, wherein the quencher is selected from the group consisting of TAMRA, Black Hole Quencher, Deep Dark Quencher, ZEN, Iowa Black FQ, Iowa Black RQ, and DABCYL.

39. A composition comprising a detectably labeled oligonucleotide probe comprising 5′ TAAATATAAATGCAAACGATGAAAACTTTGC 3′ (SEQ ID NO: 3).

40. The composition of claim 39, wherein the detectable label is a fluorescent label.

41. The composition of claim 40, wherein the fluorescent label is selected from the group consisting of fluorescein, Cy3, Cy5, Cy5.5 tetrachloro-6-car-boxyfluorescein, 2,7-dimethoxy-4,5-dichloro-6-carboxy-fluorescein, Yakima Yellow, Texas Red, TYE 563, ROX, TEX 615, TYE 665, TYE 705, and hexacholoro-6-carboxyfluorescein.

42. The composition of any one of claims 39-41, wherein the oligonucleotide probe further comprises at least one quencher.

43. The kit of claim 42, wherein the quencher is selected from the group consisting of TAMRA, Black Hole Quencher, Deep Dark Quencher, ZEN, Iowa Black FQ, Iowa Black RQ, and DABCYL.

44. A method for selecting a subject exhibiting pneumonia-like symptoms for treatment with a therapeutic agent that inhibits Legionella pneumophila, the method comprising:
(a) contacting a sample isolated from the subject with a first primer pair suitable for amplifying an ssrA target nucleic acid;
(b) contacting the sample with a second primer pair suitable for amplifying a 16S rRNA target nucleic acid;
(c) amplifying the ssrA target nucleic acid and the 16S rRNA target nucleic acid, if present; and
(d) detecting one or more amplification products produced in step (c); and
(e) selecting the subject for treatment with a therapeutic agent that inhibits Legionella pneumophila if an amplification product for the 16S rRNA target nucleic acid is detected.

45. A method of treating a subject with a Legionella pneumophila infection, the method comprising administering a therapeutic agent that inhibits Legionella pneumophila to a subject selected by the method of claim 44.

46. The method of claim 44 or 45, wherein the first primer pair comprises at least one degenerate primer.

47. The method any one of claims 44-46, wherein the first primer pair comprises a first forward primer comprising 5′ TCGACGTGGGTTGCRAAACG 3′ (SEQ ID NO: 10) or a complement thereof.

48. The method of any one of claims 44-47, wherein the first primer pair comprises a first reverse primer comprising 5′ TATGACCGTTGATTCGATACC 3′(SEQ ID NO: 2) or a complement thereof.

49. The method of any one of claims 44-48, wherein the second primer pair comprises at least one degenerate primer.

50. The method of any one of claims 44-49, wherein the second primer pair comprises a second forward primer comprising 5′ TACCTACCCTTGACATACAGTG 3′ (SEQ ID NO: 4) or a complement thereof.

51. The method of any one of claims 44-50, wherein the second primer pair comprises a second reverse primer comprising 5′ CTTCCTCCGGTTTGTCAC 3′ (SEQ ID NO: 5) or a complement thereof.

52. The method of any one of claims 44-51, further comprising contacting the biological sample with one or more oligonucleotide probes capable of specifically hybridizing to an amplification product or a complement thereof.

53. The method of claim 52, wherein the oligonucleotide probe is detectably labeled.

54. The method of claim 53, wherein the detectable label is a fluorescent label.

55. The method of claim 54, wherein the fluorescent label is selected from the group consisting of fluorescein, Cy3, Cy5, Cy5.5 tetrachloro-6-car-boxyfluorescein, 2,7-dimethoxy-4,5-dichloro-6-carboxy-fluorescein, Yakima Yellow, Texas Red, TYE 563, ROX, TEX 615, TYE 665, TYE 705, and hexacholoro-6-carboxyfluorescein.

56. The method of any one of claims 52-55, wherein the oligonucleotide probe further comprises at least one quencher.

57. The method of claim 56, wherein the quencher is selected from the group consisting of TAMRA, Black Hole Quencher, Deep Dark Quencher, ZEN, Iowa Black FQ, Iowa Black RQ, and DABCYL.

58. The method of any one of claims 52-57, wherein the oligonucleotide probe specifically hybridizes to an ssrA amplification product and wherein the oligonucleotide probe comprises 5′ TAAATATAAATGCAAACGATGAAAACTTTGC 3′(SEQ ID NO: 3) or a complement thereof.

59. The method of any one of claims 52-58, wherein the oligonucleotide probe specifically hybridizes to a 16S rRNA amplification product and wherein the oligonucleotide probe comprises 5′ CCAGCATGTGATGGTGGGGACTCTA 3′(SEQ ID NO: 6) or a complement thereof.

60. The method of any one of claims 44-59, further comprising admixing exogenous control DNA with the biological sample.

61. The method of claim 60, further comprising contacting the biological sample with a third primer pair suitable for amplification of an exogenous control target nucleic acid and amplifying the exogenous control target nucleic acid.

62. The method of claim 61, wherein the exogenous control target nucleic acid comprises SEQ ID NO: 20.

63. The method of claim 62, wherein the third primer pair consists of a third forward primer comprising 5′ GCTTCAGTACCTTCGGCTTG 3′ (SEQ ID NO: 17) and a third reverse primer comprising 5′ TTGCAGGCATCTCTGACAAC 3′ (SEQ ID NO: 18).

64. The method of claim 61 or 62, further comprising contacting the biological sample with a third oligonucleotide probe, wherein the third oligonucleotide probe is detectably labeled and comprises 5′ TGGCTCTTGGCGGTCCAGATG 3′ (SEQ ID NO: 19).

65. The method of any one of claims 44-64, wherein real-time PCR amplification is performed in a direct amplification disc in concert with an integrated thermal cycler.

66. The method of any one of claims 44-65, wherein the biological sample is a bronchoalveolar lavage sample, a bronchial wash sample, a sputum sample, a nasopharyngeal (NP) aspirate or wash sample, a nasal swab, or a bacterial isolate.

67. The method of any one of claims 44-66, wherein the therapeutic agent that inhibits Legionella pneumophila is one or more agents selected from the group consisting of fluoroquinolones, carbapenems, macrolide-antibiotics, trimethoprim-sulfamethoxazole, Legionella pneumophila-specific antibodies, and Legionella pneumophila-specific vaccines.

68. The method of claim 67, wherein the fluoroquinolones are selected from the group consisting of ciprofloxacin, gemifloxacin, levofloxacin, norfloxacin, ofloxacin, rovafloxacin, gatifloxacin, grepafloxacin, temafloxacin, lomefloxacin, sparfloxacin, enoxacin, and moxifloxacin.

69. The method of claim 67, wherein the carbapenems are selected from the group consisting of imipenem, meropenem, ertapenem, doripenem, panipenem, biapenem, razupenem (PZ-601), tebipenem, lenapenem, tomopenem, and thienpenem (Thienamycin).

70. The method of claim 67, wherein the Legionella pneumophila-specific vaccine is selected from the group consisting of whole-cell (wP) Legionella pneumophila vaccine and acellular Legionella pneumophila vaccine.

71. The method of claim 67, wherein the macrolide-antibiotics are selected from the group consisting of azithromycin (Zithromax), clarithromycin (Biaxin), erythromycin (E-Mycin, Eryc, Ery-Tab, PCE, Pediazole, Ilosone), and roxithromycin.
